(19) United States
(12) Patent Application Publication
(10) Pub. No.: US 2022/0383986 A1
(43) Pub. Date: Dec. 1, 2022

(54) Complex System for Contextual Spectrum Mask Generation Based on Quantitative Imaging

(71) Applicant: The Board of Trustees of the University of Illinois, Urbana, IL (US)

(72) Inventors: Gabriel Popescu, Urbana, IL (US); Mark A. Anastasio, Champaign, IL (US); Chenfei Hu, Urbana, IL (US); Shenghua He, Urbana, IL (US); Yuchen He, Urbana, IL (US)

(21) Appl. No.: 17/826,392

(22) Filed: May 27, 2022

Related U.S. Application Data
(60) Provisional application No. 63/194,603, filed on May 28, 2021.

(51) Int. Cl. G16B 45/00; G16B 40/10; G06T 7/00

(52) U.S. Cl. CPC G16B 45/00; G16B 40/10; G06T 7/0012; G06T 2207/10056; G06T 2207/30024

(57) ABSTRACT

Methods, apparatus, and storage medium for determining a condition of a biostructure by a neural network based on quantitative imaging data (QID) corresponding to an image of the biostructure. The method includes obtaining specific quantitative imaging data (QID) corresponding to an image of a biostructure; determining a context spectrum selection from context spectrum including a range of selectable values by: applying the specific QID to an input layer of a context-spectrum neural network, wherein the context-spectrum neural network is trained, according to a combination of focal loss and dice loss, based on previous QID and constructed context spectrum data associated with the previous QID; mapping the context spectrum selection to the image to generate a context spectrum mask for the image; and determining a condition of the biostructure based on the context spectrum mask.

### PRIORITY AND RELATED APPLICATION

This application claims priority to U.S. Provisional Application No. 63/194,603, filed May 28, 2021, which is incorporated by reference in its entirety.

### STATEMENT REGARDING FEDERALLY SPONSORED RESEARCH OR DEVELOPMENT

This invention was made with government support under R01 CA238191 and R01 GM129709 awarded by the National Institutes of Health. The government has certain rights in the invention.

### TECHNICAL FIELD

This disclosure relates to generating contextual spectrum masks for quantitative images.

### BACKGROUND

Rapid advances in biological sciences have resulted in increasing application of microscopy techniques to characterize biological samples. As an example, microscopy is in active usage in research-level and frontline medical applications. Accordingly, trillions of dollars' worth of biological research and applications are dependent on microscopy techniques. Improvements in microscopy systems will continue to improve the performance and adoption of microscopy systems.

### BRIEF DESCRIPTION OF THE DRAWINGS

The system, device, product, and/or method described below may be better understood with reference to the following drawings and description of non-limiting and non-exhaustive embodiments. The components in the drawings are not necessarily to scale. Emphasis instead is placed upon illustrating the principles of the present disclosure. The patent or application file contains at least one drawing executed in color. Copies of this patent or patent application publication with color drawing(s) will be provided by the Office upon request and payment of the necessary fee.

FIG. 1 shows an example device for contextual mask generation.

FIG. 2 shows a computer system that may be used to implement various components in an apparatus/device or various steps in a method described in the present disclosure.

FIG. 3 shows a flow diagram of an embodiment of a method in the present disclosure.

FIG. 4 shows example quantitative image data paired with an example context mask for example cells.

FIGS. 5A and 5B show schematic of the imaging system and representative results in the present disclosure.

FIGS. 6A and 6B show principle of E-U-Net training in the present disclosure.

FIG. 7 shows results of E-U-Net on testing dataset in the present disclosure.

FIG. 8 shows results of phase imaging with computation specificity (PICS) on adherent cells in the present disclosure.

FIGS. 9A and 9B show viability of cells with and without reagent stains in the present disclosure.

FIG. 10 shows an evaluation result of an E-U-Net performance in the present disclosure.

FIG. 11 shows another evaluation result of an E-U-Net performance in the present disclosure.

FIG. 12 shows a histogram of fluorescence signal ratio in an embodiment in the present disclosure.

FIG. 13 shows a pixel-wise evaluation of a trained E-U-Net in an embodiment in the present disclosure.

FIG. 14 shows results of an embodiment in the present disclosure.

FIG. 15 shows results of an embodiment in the present disclosure.

FIG. 16 shows an exemplary cells viability training of an embodiment in the present disclosure.

FIG. 17 shows results of an embodiment in the present disclosure.

FIG. 18 shows results of an embodiment in the present disclosure.

FIG. 19 shows results of an embodiment in the present disclosure.

FIG. 20 shows results of an embodiment in the present disclosure.

FIG. 21 shows results of an embodiment in the present disclosure.

FIG. 22 shows results of an embodiment in the present disclosure.

FIG. 23 shows results of an embodiment in the present disclosure.

FIG. 24 shows results of an embodiment in the present disclosure.

FIGS. 25A and 25B show a schematic of the imaging system in the present disclosure.

FIGS. 26A and 26B shows an exemplary PICS training procedure in the present disclosure.

FIG. 27 shows results of an exemplary embodiment on a test dataset in the present disclosure.

FIGS. 28A and 28B shows performance of an exemplary embodiment on a test dataset in the present disclosure.

FIGS. 29A and 29B show results of an exemplary embodiment in the present disclosure.

FIG. 30 shows statistical analysis of an exemplary embodiment on a test dataset in the present disclosure.

FIG. 31 shows an exemplary ground truth mask generation workflow of an embodiment in the present disclosure.

FIG. 32 shows performance evaluated at a pixel level of an embodiment in the present disclosure.

FIG. 33 shows an exemplary post-processing workflow of an embodiment in the present disclosure.

FIG. 34 shows an exemplary confusion matrix after merging two labels together of an embodiment in the present disclosure.

### DETAILED DESCRIPTION

The disclosed systems, devices, and methods will now be described in detail hereinafter with reference to the accompanied drawings that form a part of the present application and show, by way of illustration, examples of specific embodiments. The described systems and methods may, however, be embodied in a variety of different forms and, therefore, the claimed subject matter covered by this disclosure is intended to be construed as not being limited to any of the embodiments. This disclosure may be embodied as methods, devices, components, or systems. Accordingly, embodiments of the disclosed system and methods may, for example, take the form of hardware, software, firmware or any combination thereof.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” or “in other embodiments” as used herein does not necessarily refer to a different embodiment. It is intended, for example, that claimed subject matter may include combinations of exemplary embodiments in whole or in part. Moreover, the phrase “in one implementation”, “in another implementation”, “in some implementations”, or “in some other implementations” as used herein does not necessarily refer to the same implementation(s) or different implementation(s). It is intended, for example, that claimed subject matter may include combinations of the disclosed features from the implementations in whole or in part.

In general, terminology may be understood at least in part from usage in context. For example, terms, such as “and”, “or”, or “and/or,” as used herein may include a variety of meanings that may depend at least in part upon the context in which such terms are used. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

The present disclosure describes various embodiment for determining a condition of a biostructure according to quantitative imaging data (QID) with a neural network.

During biological research/development or medical diagnostic procedure, a condition of a biostructure may need to be analyzed and/or quantified. The biostructure may include a cell, a tissue, a cell part, an organ, or a particular cell line (e.g., HeLa cell); and the condition may include viability, cell membrane integrity, health, or cell cycle. For example, a viability analysis may classify a viability stage of a cell, including: a viable state, an injured state, or a dead state. For another example, a cell cycle analysis may classify a particular cell cycle for a cell, including: a cell growth stage (G1 phase), a deoxyribonucleic acid (DNA) synthesis stage (S phase), a second cell growth stage (G2 phase), or a mitotic stage (M phase).

Most traditional approaches for determining a condition of a biostructure rely on fluorescence microscopy to monitor the activity of proteins that are involved in the biostructure, leading to many issues/problems. For example, some issues/problems may include photobleaching, chemical toxicity, phototoxicity, weak fluorescent signals, and/or nonspecific binding. These issues/problems may impose significant limitations on its application, for example but not limited to, ability of fluorescence imaging to study live cell cultures over extended period of time.

In various embodiments in the present disclosure, quantitative phase imaging (QPI) provides a label-free imaging method for obtaining QID for a biostructure, addressing at least one of the problems/issues described above. A neural network/deep-learning network, based on the QID, can determines a condition of the biostructure. The neural network/deep-learning network in various embodiments in the present disclosure may help computationally substitute chemical stains for biostructures, extract biomarkers of interests, enhance imaging quality.

Quantitative imaging includes various imaging techniques that provide quantifiable information in addition to visual data for an image. For example, fluorescence imaging may provide information on the type and/or condition of a sample under test via usage of a dye that attaches to and/or penetrates into (e.g., biological) materials in specific circumstances. Another example, phase imaging, may use phase interference (e.g., as a comparative effect) to probe dry mass density, material transport, or other quantifiable characteristics of a sample.

In various scenarios, for a given quantitative image obtained using a given quantitative imaging (QI) technique, sources for contextual interpretation and/or contextual characterizations supported by other QI techniques may be unavailable. In an illustrative scenario, a live cell sample may be imaged using a quantitative phase imaging (QPI) that leaves the sample unharmed. However, to characterize various states of the sample it may be advantageous to have access to fluorescence imaging data in addition to (or instead of) the available QPI data. In this scenario, a challenge may entail obtaining such fluorescence imaging data without harming the live cell sample. A system that provided fluorescence imaging data and QPI data using non-destructive QPI would overcome this challenge. Further, example QI techniques may include diffraction tomography (e.g., white-light diffraction tomography) and Fourier transform light scattering.

In another illustrative scenario, one or more quantitative images may provide data to support characterization of various cell parts (or other biological structures), but the number parts or images may be too numerous for expert identification of the parts within the images to be feasible. A system the provided labelling of cell parts within the quantitative images without expert input for each image/part would overcome this challenge.

The techniques and architectures discussed herein provide solutions to the above challenges (and other challenges) by using quantitative image data (QID) as input to generate contextual masks. The generated contextual masks may provide mappings of expected context to pixels of the QID. For example, a contextual mask may indicate whether a pixel within QID depicts (e.g., at least a portion of) a particular biological structure. In an example, a contextual mask may indicate an expected fluorescence level (and/or dye concentration level) at a pixel. Providing an indication of the expected fluorescence level at a pixel may allow for a QID image (other than a fluorescent-dye-labeled image) to have the specificity of a fluorescent-dye-labeled image without imparting the harm to biological materials that is associated with some fluorescent dyes.

Further, the QID may additionally have the quantitative parameters (e.g., per-pixel quantitative data) present in the QID without mask generation. Accordingly, either QID plus a contextual mask may have more data to guide analysis of a sample than the contextual mask or the QID would have alone. In an example scenario, a contextual mask may be generated from QID were the contextual mask labels biological structures represented by pixels in the QID. The quantitative parameters for the pixels present in the QID may then be referenced against data in a structural index to characterize the biological structures based on the indications of which pixels represent which biological structures. In a real world example, QPI may be used to image spermatozoa. A contextual mask that labels the various structures of the spermatozoa may be generated. The QPI data, which may be used to determine properties such a dry mass ratios, volume, mass transport, and other quantifiable parameters may be referenced against a database of such factors indexed for viability at various stages of reproductive development. Based on the database reference, a viability determination may be made for the various spermatozoa imaged in the QPI data. Thus, the contextual mask and QPI data acquisition system may be used as an assistive-reproductive-technology (ART) system that aids in the selection of viable spermatozoa from a group of spermatozoa with varying levels of viability.

ART is a multibillion-dollar industry with applications touching various other industries including family planning and agriculture. A significant bottleneck in the industry is the reliance on human expertise and intuition to select gametes, zygotes, blastocysts, and other biological specimens from among others ensure that those in better condition are used first (e.g., to avoid millions of dollars of wasted investment on attempted reproduction using ultimately non-viable specimens). Accordingly, a contextual identification of biological structures within QID followed by quantitative characterization of those biological structures using quantitative parameters in the QID will provide a commercial advantage over existing technologies because use of contextual mask generation and quantitative parameter characterization will reduce waste in investments (both time and monetary) made in non-viable specimens. Similarly, contextual identification of biological structures within QID followed by quantitative characterization of those biological structures using quantitative parameters in the QID will provide commercial success because the reduction in waste will provide marginal value well in excess of the production and purchase costs of the system.

In various implementations, the contextual mask may be generated by providing QID as an input to neural network, which provides the contextual mask as an output. The neural network may be trained using input-result pairs. The input-result pairs may be formed using QID of the desired input type captured from test samples and constructed context masks that include the desired output context for the test samples. The constructed context masks may refer to context masks that are generated using the nominal techniques for obtaining the desired output context. For example, a constructed context mask including fluorescence-contrast image may be obtained using fluorescence-contrast imaging. In an example, a constructed context mask including expert-identified biological structure indications may be obtained using human expert input. The input-result pairs may be used to adjust the interneuron weights within neural network during the training process. After training, the neural network may be used to compare current QID to the training QID used in training process via the interneuron weights. This comparison then generates a context mask (e.g., a simulated context mask, a mask with expected contextual values, or other) without use of the nominal technique. Thus, using the trained neural network, a context mask with the desired output context may be obtained even when then performance of nominal technique is undesirable (e.g., because of harmful effects), impracticable (e.g., because of limited expert capacity/availability), or otherwise unavailable.

In various implementations, generation of a contextual mask based on QID may be analogous to performing image transformation operation on the QID. Accordingly, various machine-learning techniques to support image transformation operations may be used (e.g., including classification algorithms, convolutional neural networks, generative adversarial networks (GAN), or other machine learning techniques to support image transformation/translation). In various implementations, a “U-net” type convolutional neural network may be used.

In some implementations, subtle differences in sample makeup may indicate differences in sample condition. For example, some dye contrast techniques may provide contrast allowing cells with similar visible appearances to be distinguished with regard to their viability state. For example, a spectrum-like dye analysis may allow classification of cells into live (viable), injured, and dead classifications. In various implementations, QID may include information that may support similar spectrum classifications (e.g., which may use continuum or near continuum image data analysis to classify samples). A context-spectrum neural network (e.g., may (in some cases) use an EfficientNet design in conjunction with a transfer learning process (as discussed in the drawings, examples, and claims below) may be used to generate contextual masks and/or context spectrum masks. Further, context-spectrum neural networks may be used with e.g., capture subsystems to capture QID or other devices and/or subsystems discussed below for training and/or analysis purposes.

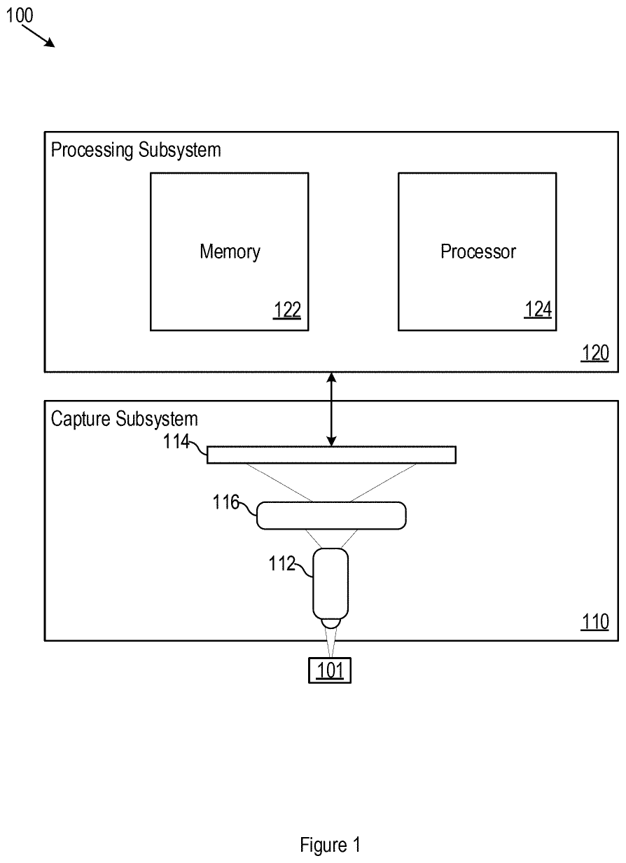

Referring now to FIG. 1 an example device 100 for contextual mask generation is shown. The example device 100 may include a capture subsystem 110 for capture of QID images of a sample 101. In the example, the capture subsystem includes an objective 112 and a pixel array 114. In various implementations, the capture subsystem 110 may include a processing optic 116 that may generate a comparative effect (e.g., a tomographical effect, a differential interference effect, a Hoffman contrast effect, a phase interference effect, Fourier transform effect, or other comparative effect) that may allow for the capture of QID (beyond visual data). In some implementations, QID may be obtained from dyes, sample processing, or other techniques in lieu of a comparative effect.

The pixel array 114 may be positioned at an image plane of the objective 112 and/or a plane of the comparative effect generated via the processing optic 116. The pixel array 114 may include a photosensitive array such as a charge-coupled device (CCD), complimentary metal-oxide-semiconductor (CMOS) sensor, or other sensor array.

The processing optic 116 may include active and/or passive optics that may generate a comparative effect from light rays focused trough the objective 112. For example, in a QPI-based system on gradient light inference microscopy (GLIM) the processing optic 116 may include a prism (e.g., a Wollaston prism, a Normanski prism, or other prism) that generates two replicas of an image field with a predetermined phase shift between them. In an example based on spatial light interference microscopy, the processing optic 116 may include a spatial light modulator (SLM) between two Fourier transforming optics (e.g., lenses, gratings, or other Fourier transforming optics). The controllable pixel elements of the SLM may be used to place selected phase-shifts on frequency components making up a particular light ray. Other comparative effects and corresponding processing optics 116 may be used.

The example device 100 may further include a processing subsystem 120. The processing subsystem may include memory 122 and a hardware-based processor 124. The memory 122 may store raw pixel data from the pixel array 114. The memory may further store QID determined from the raw pixel data and/or instructions for processing the raw pixel data to obtain the QID. Thus, the QID may include pixel values including visual data from the raw pixel data and/or quantitative parameters derived from analysis of the comparative effect and the pixel values of the raw pixel data. The memory may store a neural network (or other machine learning protocol) to generate a context mask based on the QID. The memory may store the context mask after generation.

In some distributed implementations, not shown here, the processing subsystem 120 (or portions thereof) may be physically removed from the capture subsystem 110. Accordingly, the processing subsystem 120 may further include networking hardware (e.g., as discussed with respect to context computation environment (CCE) 500 below) that may receive raw pixel data and/or QID in a remotely captured and/or partially remotely-pre-processed form.

The processor 124 may execute instructions stored on the memory to derive quantitative parameters from the raw pixel data. Further, the processor 124 may execute the neural network (or other machine learning protocol) stored on the memory 122 to generate the context mask.

In some implementations, the example device 100 may support a training mode where constructed context masks and training QID are obtained contemporaneously (in some cases simultaneously). For example, a test sample may be prepared with contrast dye and then imaged using the capture subsystem 110. The processing subsystem may use fluorescence intensities present in the raw pixel data as a constructed context mask. In some cases, the fluorescence intensities present in the raw pixel data may be cancelled (e.g., through a normalization process, through symmetries in the analysis of the comparative effect, or through another cancellation effect of the QID derivation) during extraction of the quantitative parameters. Accordingly, in some cases, a constructed context mask may be obtained from the overlapping raw pixel data (e.g., the same data, a superset, a subset, or other partial overlap) with that from which the QID is obtained.

For the training mode, the memory may further include training protocols for the neural network (or other machine learning protocol). For example, the protocol may instruct that the weights of the neural network be adjusted over a determined number of training epochs using a determined number of input-result training pairs obtained from the captured constructed masks and derived QID.

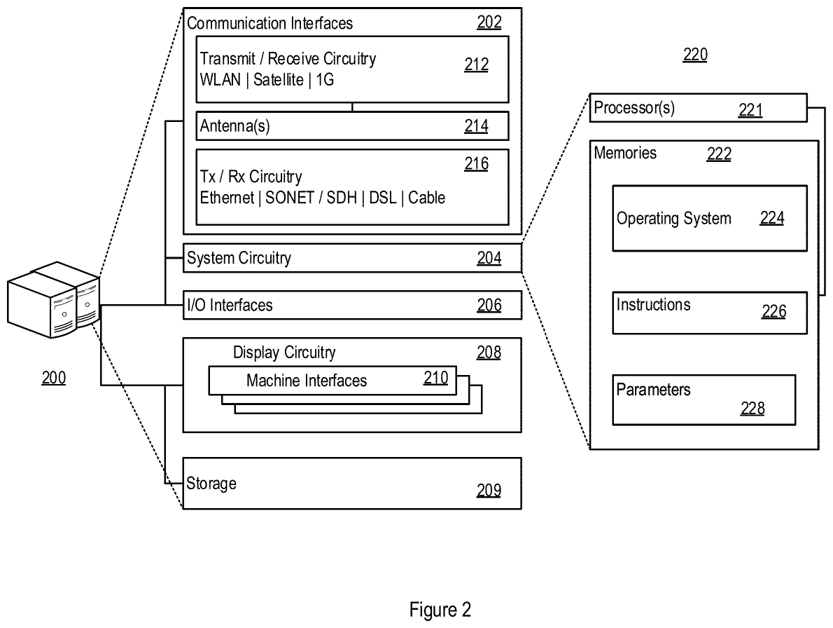

FIG. 2 shows an exemplary electronic device/apparatus (e.g., the processing subsystem 120) for obtaining QID corresponding to a biostructure and/or determining a condition of the biostructure. The electronic device/apparatus may include a computer system 200 for implementing one or more steps in various embodiments of the present disclosure. The computer system 200 may include communication interfaces 202, system circuitry 204, input/output (I/O) interfaces 206, storage 209, and display circuitry 208 that generates machine interfaces 210 locally or for remote display, e.g., in a web browser running on a local or remote machine. For one example, the computer system 200 may communicate with one or more instrument (e.g., a capture subsystem 110, as shown in FIG. 1). For another example, the computer system 200 may not directly communicate with a capture subsystem, but indirectly obtain QID of a biostructure (e.g., from a data server or a storage device), and then may process the QID to determining a condition of the biostructure using a neural network as described in the present disclosure.

The machine interfaces 210 and the I/O interfaces 206 may include GUIs, touch sensitive displays, voice or facial recognition inputs, buttons, switches, speakers and other user interface elements. Additional examples of the I/O interfaces 206 include microphones, video and still image cameras, headset and microphone input/output jacks, Universal Serial Bus (USB) connectors, general purpose digital interface (GPIB), peripheral component interconnect (PCI), PCI extensions for instrumentation (PXI), memory card slots, and other types of inputs. The I/O interfaces 206 may further include magnetic or optical media interfaces (e.g., a CDROM or DVD drive), serial and parallel bus interfaces, and keyboard and mouse interfaces.

The communication interfaces 202 may include wireless transmitters and receivers (“transceivers”) 212 and any antennas 214 used by the transmitting and receiving circuitry of the transceivers 212. The transceivers 212 and antennas 214 may support Wi-Fi network communications, for instance, under any version of IEEE 802.11, e.g., 802.11n or 802.11ac. The communication interfaces 202 may also include wireline transceivers 216. The wireline transceivers 216 may provide physical layer interfaces for any of a wide range of communication protocols, such as any type of Ethernet, data over cable service interface specification (DOCSIS), digital subscriber line (DSL), Synchronous Optical Network (SONET), or other protocol.

The storage 209 may be used to store various initial, intermediate, or final data or model for implementing the embodiment for determining at least one reaction condition. These data corpus may alternatively be stored in a database 118. In one implementation, the storage 209 of the computer system 200 may be integral with a database. The storage 209 may be centralized or distributed, and may be local or remote to the computer system 200. For example, the storage 209 may be hosted remotely by a cloud computing service provider.

The system circuitry 204 may include hardware, software, firmware, or other circuitry in any combination. The system circuitry 204 may be implemented, for example, with one or more systems on a chip (SoC), application specific integrated circuits (ASIC), microprocessors, discrete analog and digital circuits, and other circuitry.

For example, at least some of the system circuitry 204 may be implemented as processing circuitry 220. The processing circuitry 220 may include one or more processors 221 and memories 222. The memories 222 stores, for example, control instructions 226, parameters 228, and/or an operating system 224. The control instructions 226, for example may include instructions for implementing various components of the embodiment for determining at least one reaction condition. In one implementation, the instruction processors 221 execute the control instructions 226 and the operating system 224 to carry out any desired functionality related to the embodiment.

The present disclosure describes various embodiments of methods and/or apparatus for determining a condition of a biostructure based on QID corresponding to an image of the biostructure, which may include or be implemented by an electric device/system as shown in FIG. 2.

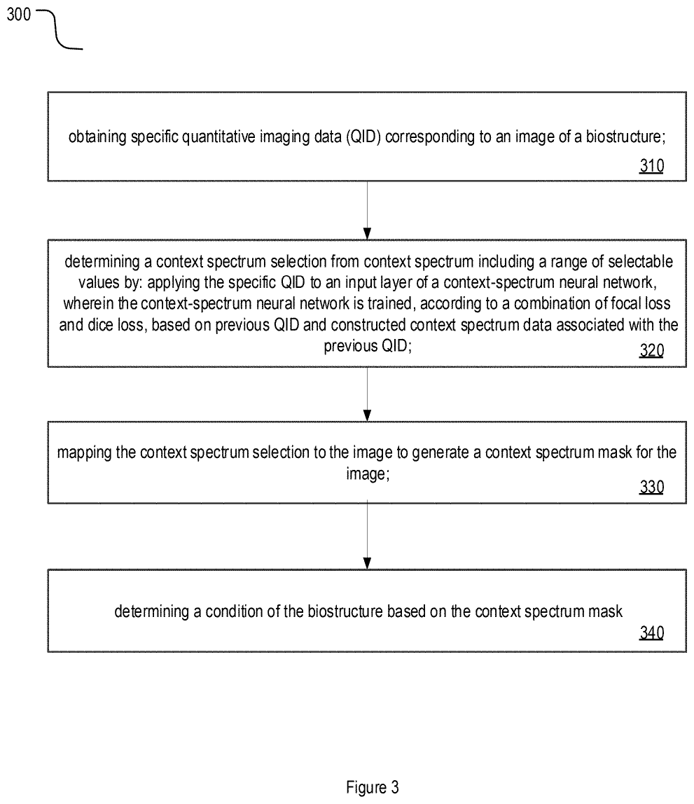

Referring to FIG. 3, the present disclosure describes various embodiments of a method 300 for determining a condition of a biostructure based on QID corresponding to an image of the biostructure. The method 300 may include a portion or all of the following steps: step 310, obtaining specific quantitative imaging data (QID) corresponding to an image of a biostructure; step 320, determining a context spectrum selection from context spectrum including a range of selectable values by: applying the specific QID to an input layer of a context-spectrum neural network, wherein the context-spectrum neural network is trained, according to a combination of focal loss and dice loss, based on previous QID and constructed context spectrum data associated with the previous QID; step 330, mapping the context spectrum selection to the image to generate a context spectrum mask for the image; and/or step 340, determining a condition of the biostructure based on the context spectrum mask. In some implementations, the context-spectrum neural network may perform both step 320 and step 330.

In some implementations, the previous QID are obtained corresponding to an image of a second biostructure; and/or the constructed context spectrum data comprises a ground truth condition of the second biostructure.

In some implementations, the context-spectrum neural network comprises an EfficientNet Unet comprising one or more first layers for adapting a vector size to operational size for another layer of the EfficientNet Unet.

In various embodiments in the present disclosure, EfficientNets refers to a family of deep convolutional neural networks that possess a powerful capacity of feature extraction but require much fewer network parameters compared to other state-of-the-art network architectures, such VGG-Net, ResNet, Mask R-CNN, etc. The EfficientNet family may include eight network architectures, from EfficientNet-B0 to EfficientNetB7, with an increasing network complexity. EfficientNet-B3 and EfficientNet-B7 were selected for training E-U-Net on HeLa cell images and CHO cell images, respectively, considering they yields the most accurate segmentation performance on the validation set among all the eight EfficientNets.

In some implementations, the biostructure comprises at least one of the following: a cell, a tissue, a cell part, an organ, or a HeLa cell.

In some implementations, the condition of the biostructure comprises at least one of the following: viability, cell membrane integrity, health, or cell cycle.

In some implementations, the context spectrum comprises a continuum or near continuum of selectable states.

In some implementations, the condition of the biostructure comprises one of a viable state, an injured state, or a dead state; or the condition of the biostructure comprises one of a cell growth stage (G1 phase), a deoxyribonucleic acid (DNA) synthesis stage (S phase), or a cell growth/mitotic stage (G2/M phase).

Various embodiments in the present disclosure may include one or more non-limiting examples of context mask generation logic (CMGL) and/or training logic (TL). More detailed description is included in U.S. application Ser. No. 17/178,486, filed on Feb. 18, 2021 by the same Applicant as the present application, which is incorporated herein by reference in its entirety.

The CMGL may compare the QID to previous QID via application of the QID to the neural network. The neural network is trained using previous QID of the same type such that application of the “specific” QID being applied currently. Accordingly, processing of the specific QID using the neural network (and its interneuron weights) effects a comparison of similarities and differences between the specific QID and the previous QID. Based on those similarities and differences a specific context mask is generated for the specific QID.

The CMGL may apply the generated context mask to the QID. The application of the context mask to the QID may provide context information that may complement characterization/analysis of the source sample. For example, the context mask may increase the contrast visible in the image used to represent the QID. In another example, the context mask may provide indications of expected dye concentrations (if a contrast dye were applied) at the pixels within the QID. The expected dye concentrations may indication biological (or other material) structure type, health, or other status or classification. The context mask may provide simulated expert input. For example, the context mask indicate which pixels within the QID represent which biological structures. The context mask may provide context that would otherwise be obtained through a biologically-destructive (e.g., biological sample harming or killing) process using the QID which in some cases may be obtained through a non-destructive process.

In various implementations, a TL may obtain training QID, and obtain a constructed mask. Using the training QID and corresponding constructed mask, the TL may form an input-result pair. The TL may apply the input-result pair to the neural network to adjust interneuron weights. In various implementations, determination of the adjustment to the interneuron weights may include determining a deviation between the constructed context mask and simulated context mask generated by the neural network in its current state. In various implementations, the deviation may be calculated as a loss function, which may be iteratively reduced (e.g., over multiple training epochs) using an optimization function. Various example optimization functions for neural network training may include a least squares algorithm, a gradient descent algorithm, differential algorithm, a direct search algorithm, a stochastic algorithm, or other search algorithm.

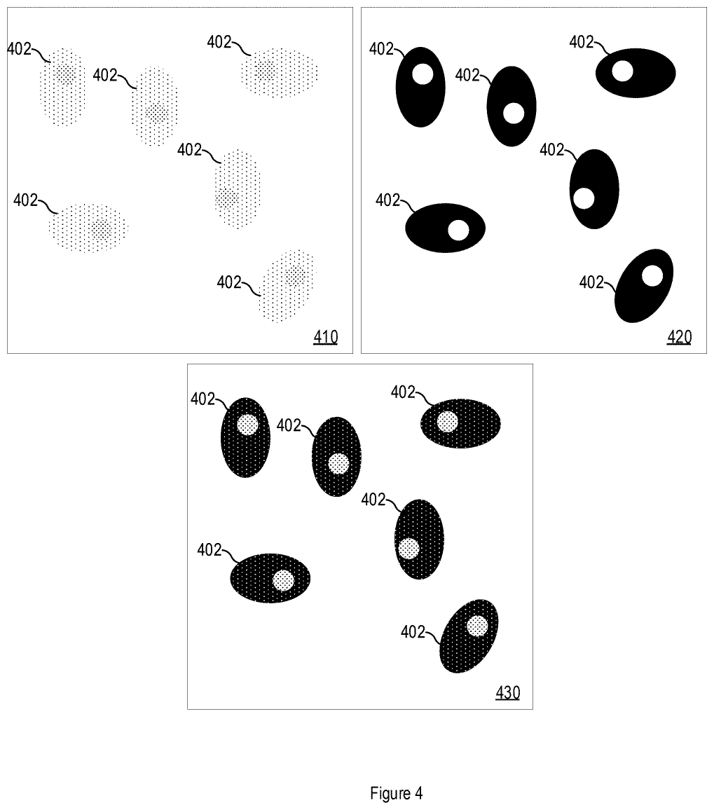

FIG. 4 shows example QID 410 paired with an example context mask 420 for example cells 402. The example QID 410 shows a density quantitative parameter (e.g., via the density of dots shown). However, in the example QID 410 low visual contrast inhibits ease of interpretation of the QID 410. The example context mask 420 provides tagging for the cell nucleus (white) and other portions (black). The combination QID/context 430 provides the density quantitative parameter mapped onto the tagging facilitating quantitative analysis of the cell structures.

The present disclosure describes a few non-limiting embodiments for determining a condition of a biostructure based on QID corresponding to an image of the biostructure: one embodiment includes a live-dead assay on unlabeled cells using phase imaging with computational specificity; and another embodiment includes a cell cycle stage classification using phase imaging with computational specificity. The embodiments and/or example implementations below are intended to be illustrative embodiments and/or examples of the techniques and architectures discussed above. The example implementations are not intended to constrain the above techniques and architectures to particular features and/or examples but rather demonstrate real world implementations of the above techniques and architectures. Further, the features discussed in conjunction with the various example implementations below may be individually (or in virtually any grouping) incorporated into various implementations of the techniques and architectures discussed above with or without others of the features present in the various example implementations below.

### Embodiment: Live-Dead Assay on Unlabeled Cells Using Phase Imaging with Computational Specificity

Existing approaches to evaluate cell viability involve cell staining with chemical reagents. However, this step of exogenous staining makes these methods undesirable for rapid, nondestructive and long-term investigation. The present disclosure describes instantaneous viability assessment of unlabeled cells using phase imaging with computation specificity (PICS). This new concept utilizes deep learning techniques to compute viability markers associated with the specimen measured by label-free quantitative phase imaging. Demonstrated on different live cell cultures, the proposed method reports approximately 95% accuracy in identifying live and dead cells. The evolution of the cell dry mass and projected area for the labelled and unlabeled populations reveal that the viability reagents decrease viability. The nondestructive approach presented here may find a broad range of applications, from monitoring the production of biopharmaceuticals, to assessing the effectiveness of cancer treatments.

Rapid and accurate estimation of viability of biological cells is important for assessing the impact of drugs, physical or chemical stimulants, and other potential factors in cell function. The existing methods to evaluate cell viability commonly require mixing a population of cells with reagents to convert a substrate to a colored or fluorescent product. For instance, using membrane integrity as an indicator, the live and dead cells can be separated by trypan blue exclusion assay, where only nonviable cells are stained and appear as a distinctive blue color under a microscope. MTT or XTT assay estimates the viability of a cell population by measuring the optical absorbance caused by formazan concentration due to alteration in mitochondrial activity. Starting in the 1970s, fluorescence imaging has developed as a more accurate, faster, and reliable method to determine cell viability. Similar to the principle of trypan blue test, this method identifies individual nonviable cells by using fluorescent reagents only taken up by cells that lost their membrane permeability barrier. Unfortunately, the step of exogenous labeling generally requires some incubation time for optimal staining intensity, making all these methods difficult for quick evaluation. Importantly, the toxicity introduced by stains eventually kills the cells and, thus, prevents the long-term investigation.

Quantitative phase imaging (QPI) is a label-free modality that has gained significant interest due to its broad range of potential biomedical applications. QPI measures the optical phase delay across the specimen as an intrinsic contrast mechanism, and thus, allows visualizing transparent specimen (i.e., cells and thin tissue slices) with nanoscale sensitivity, which makes this modality particularly useful for nondestructive investigations of cell dynamics (i.e. growth, proliferation, and mass transport) in both 2D and 3D. In addition, the optical phase delay is linearly related to the non-aqueous content in cells (referred to as dry mass), which directly yields biophysical properties of the sample of interest. More recently, with the concomitant advances in deep learning, there may be exciting new avenues for label-free imaging. In 2018, Google presented “in silico labeling”, a deep learning based approach that can predict fluorescent labels from transmitted-light (bright field and phase contrast) images of unlabeled samples. Around the same time, researchers from the Allen Institute showed that individual subcellular structure such as DNA, cell membrane, and mitochondria can be obtained computationally from bright-field images. As a QPI map quantitatively encodes structure and biophysical information, it is possible to apply deep learning techniques to extract subcellular structures, perform signal reconstruction, correct image artifacts, convert QPI data into virtually stained or fluorescent images, and diagnose and classify various specimens.

The present disclosure shows that rapid viability assay can be conducted in a label-free manner using spatial light interference microscopy (SLIM), a highly sensitive QPI method, and deep learning. The concept of a newly-developed phase imaging with computational specificity (PICS) is applied to digitally stain for the live and dead markers. Demonstrated on live adherent HeLa and CHO cell cultures, the viability of individual cell measured with SLIM is predicted by using a joint EfficientNet and transfer learning strategy. Using the standard fluorescent viability imaging as ground truth, the trained neural network classifies the viable state of individual cell with 95% accuracy. Furthermore, by tracking the cell morphology over time, unstained HeLa cells show significantly higher viability compared to the cells stained with viability reagents. These findings suggest that PICS method enables rapid, nondestructive, and unbiased cell viability assessment, potentially valuable to a broad range of biomedical problems, from drug testing to production of biopharmaceuticals.

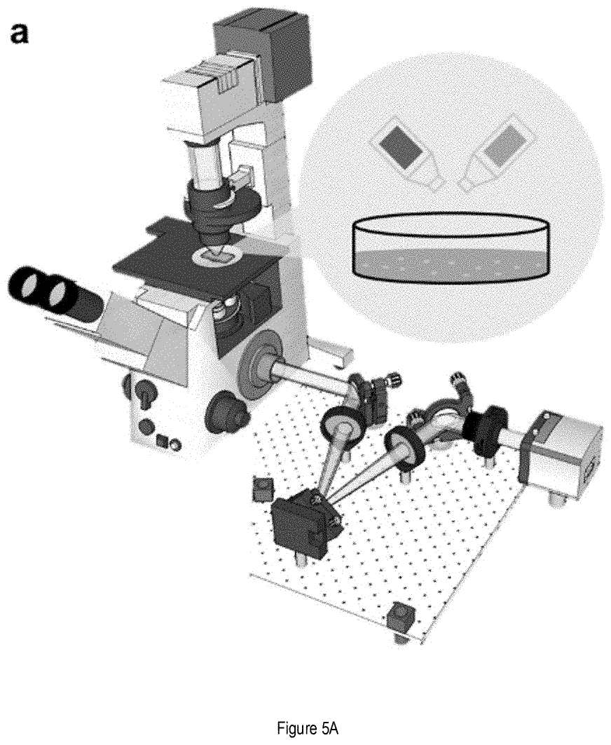

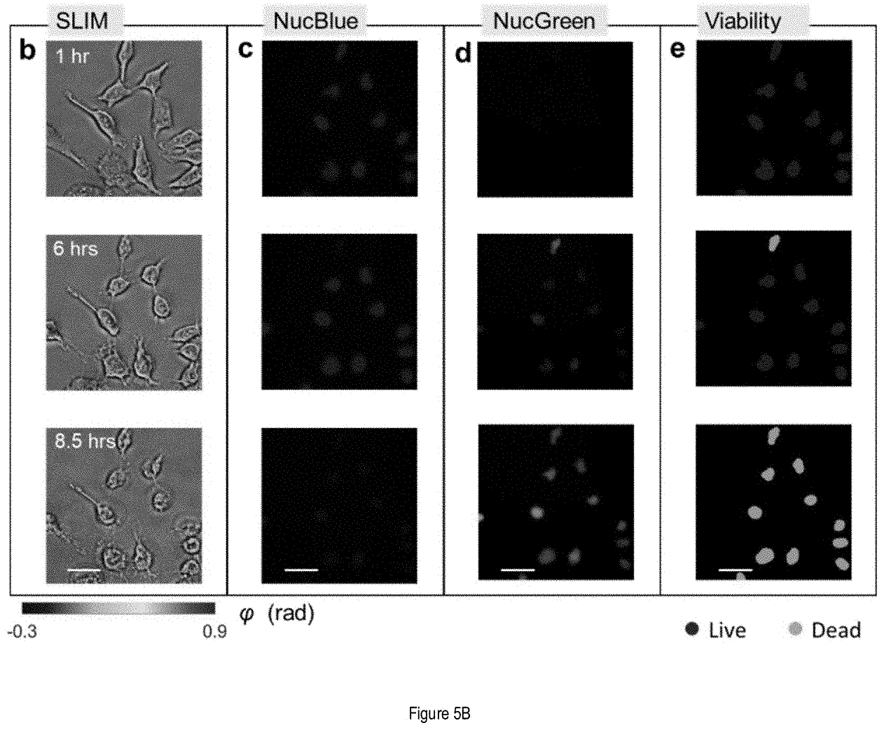

The procedure of image acquisition is summarized in FIGS. 5A and 5B. Spatial light interference microscopy (SLIM) is employed to measure quantitative phase map of cells in vitro. The system is built by attaching a SLIM module (e.g., CellVista SLIM Pro, Phi Optics, Inc.) to the output port of an existing phase-contrast microscope (FIG. 5A). By modulating the optical phase delay between the incident and the scattered field, a quantitative phase map is retrieved from four intensity images via phase-shifting interferometry. SLIM employs a broadband LED as illumination source and common-path imaging architecture, which yields sub-nanometer sensitivity to optical pathlength changes and high temporal stability. By switching to epi-illumination, the optical path of SLIM is also used to record the fluorescent signals over the same field of view. Detailed information about the microscope configuration can be found in Methods.

To demonstrate the feasibility of the proposed method, live cell cultures are imaged and analyzed. Before imaging, 40 micro-liter (μL) of each cell-viability-assay reagent (e.g., ReadyProbes Cells Viability Imaging Kit, Thermofisher) was added into 1 ml growth media, and the cells were then incubated for approximately 15 minutes to achieve optimal staining intensity. The viability-assay kit contains two fluorescently labeled reagents: NucBlue (the “live” reagent) combines with the nuclei of all cells and can be imaged with a DAPI fluorescent filter set, and NucGreen (the “dead” reagent) stains the nuclei of cells with compromised membrane integrity, which is imaged with a FITC filter set. In this assay, live cells produce blue-fluorescent signal; dead cells emit both green and blue fluorescence; The procedure of cell culture preparation may be found in some of following paragraphs.

After staining, the sample was transferred to the microscope stage, and measured by SLIM and epi-fluorescence microscopy. In order to generate a heterogeneous cell distribution that shifts from predominantly alive to mostly dead cells, the imaging was performed under room conditions, such that the low-temperature and imbalanced pH level in the media would adversely injure the cells and eventually cause necrosis. Recording one measurement every 30 or 60 minutes, the entire imaging process lasted for approximately 10 hours. This experiment was repeated four times to capture the variability among different batches. FIG. 5B shows the SLIM images of HeLa cells measured at t=1 hour, 6, and 8.5 hours, respectively, and the corresponding fluorescent measurements are shown in c and d. The results in FIG. 5B show that the adverse environmental condition continues injuring the cell, where blebbing and membrane disruption could be observed during cell death. The QPI measurements agree with the results reported in previous literature. On the other hand, these morphological alterations are correlated with the changes in fluorescence signals, where the intensity of NucGreen (“dead” fluorescent channel) continuously increases, as cells transit to dead states. By comparing the relative intensity between NucGreen and NucBlue signals, semantic segmentation maps can be generated to label individual cell as either live or dead, as shown in e in FIG. 5B. The procedure of generating the semantic maps may be found in some of the following paragraphs. All collected image sequences were combined to form dataset for PICS training and testing, where each sequence is a time-lapse recording of cells from live to dead states. Then the sequences are randomly split with a ratio of approximately 6:1:1, to obtain training, validation, and testing dataset, respectively. Instead of splitting by frame, training dataset is generated by dividing image sequences to ensure fair generalization. In addition, data across all measurements are combined to take underrepresented cellular activities into account, which makes the purposed method generalizable.

FIGS. 5A and 5B show schematic of the imaging system and representative results. In FIG. 5A, CellVista SLIM Pro microscope (Phi Optics, Inc.) consists of an existing phase contrast microscope and an external module attached to the output port. By switching between transmission and reflection excitation, both SLIM and co-localized fluorescence images can be recorded via the same optical path. Before time-lapse imaging started, fluorescence viability reagents were mixed with HeLa cell culture. In FIG. 5B, b. Representative SLIM measurements of HeLa cell at 1, 6, and 8.5 hours. c. NucBlue fluorescent signals of the live viability reagent. d. NucGreen fluorescent signals of the dead viability reagents measured by a FITC filter. e. Viability states of the individual cells. Scale bars represents 50 microns.

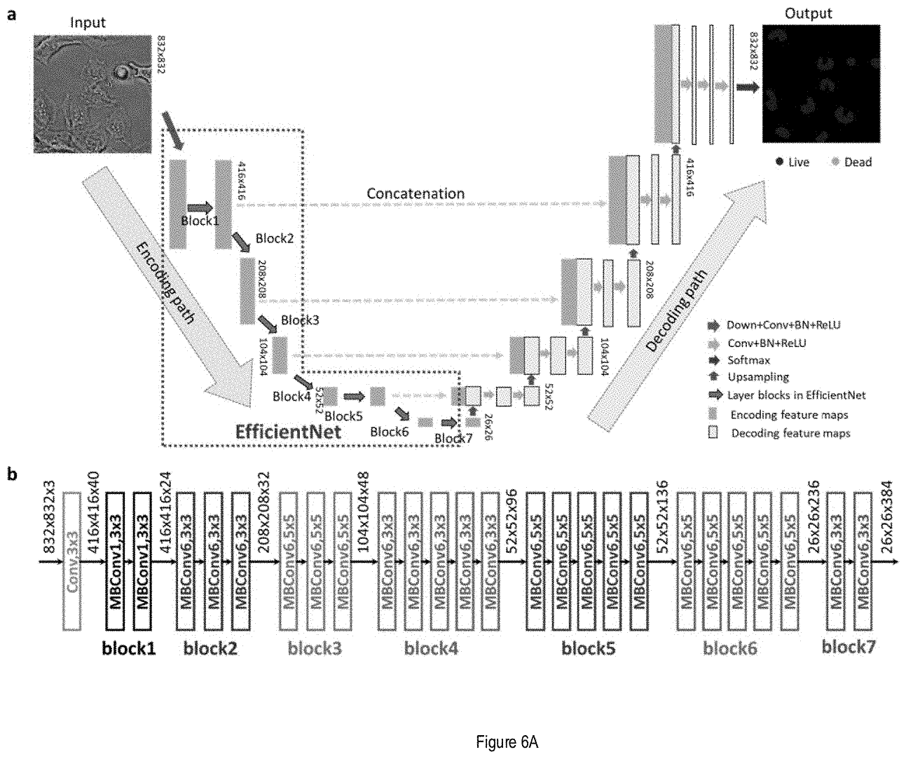

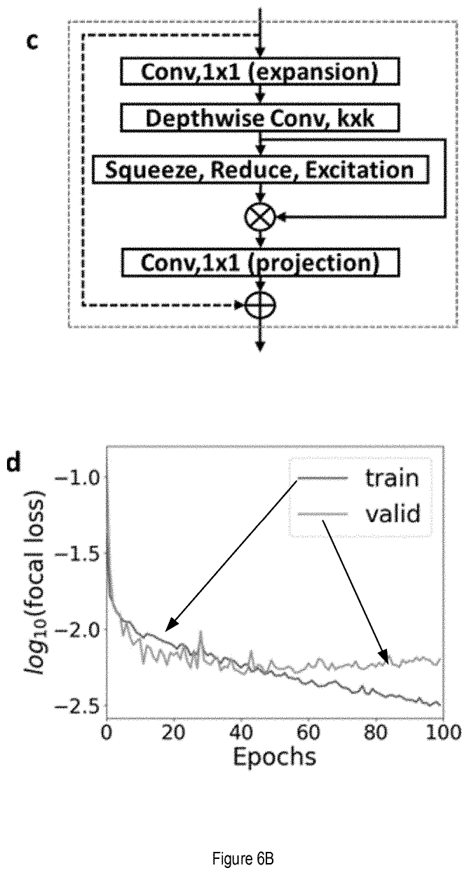

With fluorescence-based semantic maps as ground truth, a deep neural network was trained to assign “live”, “dead”, or background labels to pixels in the input SLIM images. a U-Net based on EfficientNet (E-U-Net) is employed, with its architecture shown in FIG. 6A. Compared to conventional U-Nets, the E-U-Net uses EfficientNet, a powerful network of relatively lower complexity, as the encoding part. This architecture allows for learning an efficient and accurate end-to-end segmentation model, while avoiding training a very complex network. The network was trained using a transfer learning strategy with a finite training set. At first, the EfficientNet of E-U-Net (the encoding part) was pre-trained for image classification on a publicly available dataset ImageNet. The entire E-U-Net was then further fine-tuned for a semantic segmentation task by using labeled SLIM images from training and validation set.

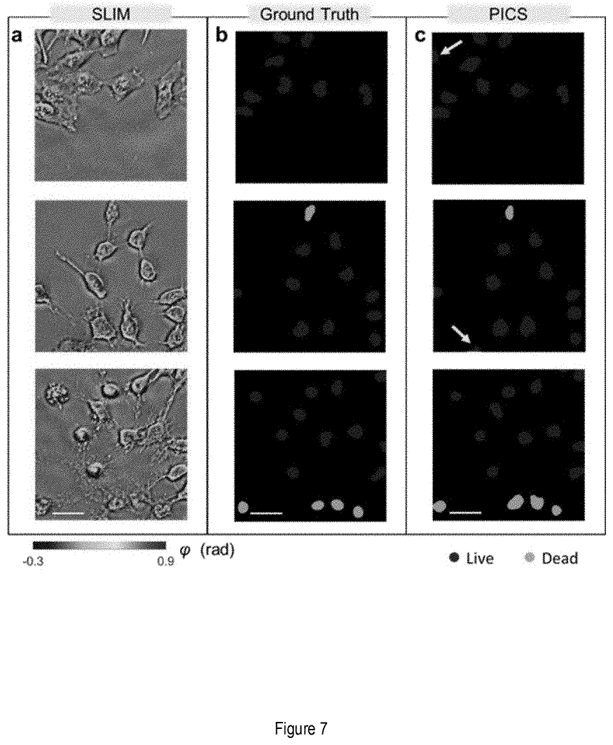

The network training was performed by updating the weights of parameters in the E-U-Net using an Adam optimizer to minimize a loss function that is computed in the training set. More details about the EfficientNet module and loss function may be found in other paragraphs in the present disclosure. The network was trained for 100 epochs. At the end of each epoch, the loss function related to the being-trained network was evaluated, and the weights that yielded the lowest loss on the validation set were selected for the E-U-Net model. In FIG. 7B, panel d shows training and validation loss vs. number of epochs, using 899 and 199 labeled images as training and validation dataset. FIGS. 6A and 6B present more details about the E-U-Net architecture and network training.

FIGS. 6A and 6B shows principle of E-U-Net training. In FIG. 6A, a. The E-U-Net. architecture includes an EfficientNet as the encoding path and five stages of decoding. The E-U-Net includes a Down+Conv+BN+ReLU block and 7 other blocks. The Down-Conv-BN-ReLU block represents a chain of down-sampling layer, convolutional layer, batch normalization layer, and ReLU layer. Similarly, the Conv+BN+ReLU is a chain of convolutional layer, batch normalization layer, and ReLU layer. b The network architecture of EfficientNet-B3. Different blocks are marked in different colors. They correspond to the layer blocks of EfficientNet in a. In FIG. 6B, c. The major layers inside the MBConvX module. X=1 and X=6 indicate the ReLU and ReLU6 are used in the module, respectively. The skip connection between the input and output of the module is not used in the first MBConvX module in each layer block. d. Training and validation loss vs epochs plotted in the log scale.

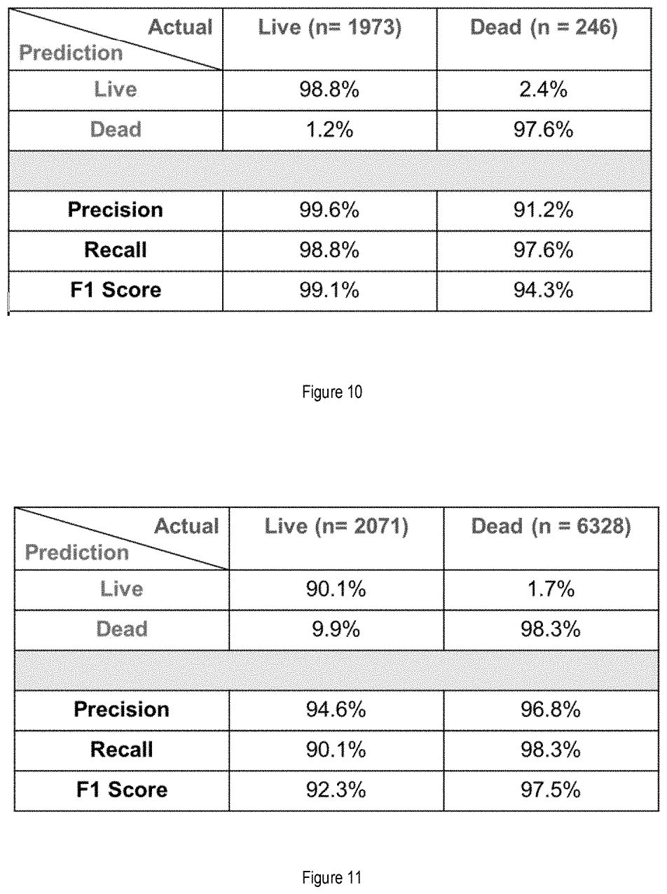

To demonstrate the performance of phase imaging with computational specificity (PICS) as a label-free live/dead assay, the trained network was applied to 200 SLIM images not used in training and validation. In FIG. 7, panel a shows the three representative testing phase maps, whereas corresponding ground truth and PICS prediction are shown in panel b and panel c, respectively. This direct comparison indicates that PICS successfully classifies the cell states. Most often, the incorrect predictions were caused by cells located at the boundary of FOV, where only a portion of their cell bodies were measured by SLIM. Finally, PICS may fail when cells become detached from the well plates. In this situation, the suspended cells appear out of focus, which gives rise to inaccurate prediction. As reported in previous publications, the conventional deep learning evaluation metrics focus on assessing pixel-wise segmentation accuracy, which overlooks some biologically relevant instances. Here, an object-based evaluation metric was adopted, which relies on comparing the dominant semantic label between the predicted cell nuclei and the ground truth for individual nucleus. The confusion matrix and the corresponding evaluation (e.g., precision, recall and F1-score) are shown in FIG. 10.

A comparison with standard pixel-wise evaluation and procedure of object-based evaluation may be performed. The entries of the confusion matrix are normalized with respect to the number of cells in each category. Using the average F1 score across all categories as an indicator of the overall performance, this PICS strategy reports a 96.7% confidence in distinguish individual live and dead HeLa cells.

Chinese hamster ovary (CHO) cells are often used for recombinant protein production, as it received U.S. FDA approval for bio-therapeutic protein production. Here, it's demonstrated that the label-free viability assay approach is applicable to other cell lines of interest in pharmaceutical applications. CHO cells were plated on a glass bottom 6-well plate for optimal confluency. In addition to NucBlue/NucGreen staining, 1 μM of staurosporine (apoptotic inducing reagent) solution was added to the culture medium. This potent reagent permeates cell membrane and disrupts protein kinase, cAMP, and lead to apoptosis in 4-6 hours. The cells were then measured by SLIM and epi-fluorescence microscopy. The cells were maintained in regular incubation condition (37° C. and 5% concentration of CO₂) throughout the experiment. In addition, it is verified that the cells were not affected by necrosis and lytic cell death. After image acquisition, E-U-Net (EfficientNet-B7) training was immediately followed. In the training process, 1536 labeled SLIM images and 288 labeled SLIM images were used for network training and validation, respectively. The structure of EfficientNet-B7, training and validation loss can be found. The trained E-U-net was finally applied to 288 unseen testing images to test the performance of dead/viability assay. The procedure of imaging, ground truth generation, and training were consistent with the previous experiments.

FIG. 7 shows results of E-U-Net on testing dataset. In panel a, representative SLIM measurements of HeLa cells not used during training. In panel b, the ground truth for viability of frames corresponding to a. In panel c, the PICS prediction shows high level accuracy in segmenting the nuclear regions and inferring viability states. The arrows indicate the inconsistence between ground truth and PICS prediction caused by the cells located at the edge of the FOV are subject to inference error. Scale bars represents 50 microns.

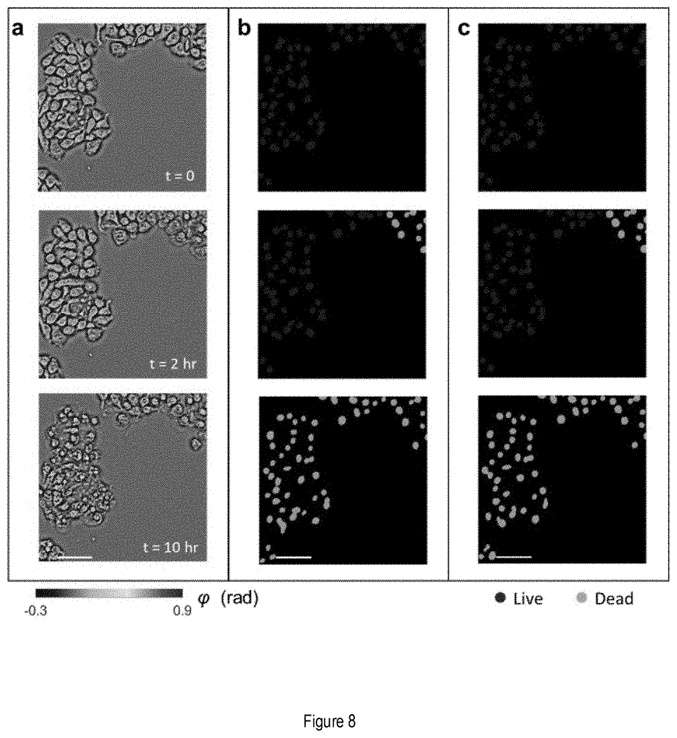

In FIG. 8, panel a shows the time-lapse SLIM image of CHO cells measured at t=0, 2, and 10 hours after adding apoptosis reagent, and the corresponding viability map determined by fluorescence signal and PICS are plotted in panel b and panel c, respectively. In contrast to necrosis, the cell bodies became gradually fragmented during apoptosis. The visual comparison suggests that PICS yields good performance in extracting cell nucleus and predicting its viable state. Running an evaluation on individual cells, as shown in FIG. 11, the network gives an average F-1 score of 94.9%. Again, the inaccurate prediction is mainly caused by cells at the boundary of the FOV. It's also found rare cases where cells show features of cells death at early stage, but it was identified as live by traditional fluorometric evaluation. Furthermore, because most of the cells stay adherent, the PICS accuracy was not affected by cell confluence, as indicated by the evaluation metrics under different confluence levels.

FIG. 8 shows results of PICS on adherent CHO cells. In panel a, time-lapse SLIM measurements of CHO cells measured at t=0, 2, and 10 hours. The data was not used during training or validation. In panel b, the ground truth for viability of frames corresponding to a. In panel c, the PICS prediction shows high level accuracy in segmenting the nuclear regions and inferring viability states. Scale bars represents 50 microns.

Performing viability assays on unlabeled cells essentially circumvents the cell injury effect caused by exogenous staining and produces an unbiased evaluation. To demonstrate this feature on a different cell type, a fresh HeLa cell culture was prepared in a 6-well plate, transferred to the microscope stage, and maintained under room conditions. Half of the wells were mixed with viability assay reagents, where the viability was determined by both PICS and fluorescence imaging. The remaining wells did not contain reagents, such that the viability of these cells was only evaluated by PICS. The procedure of cell preparation, staining, and microscope settings were consistent with the previous experiments. Measurements were took every 30 minutes, and the entire experiment lasted for 12 hours.

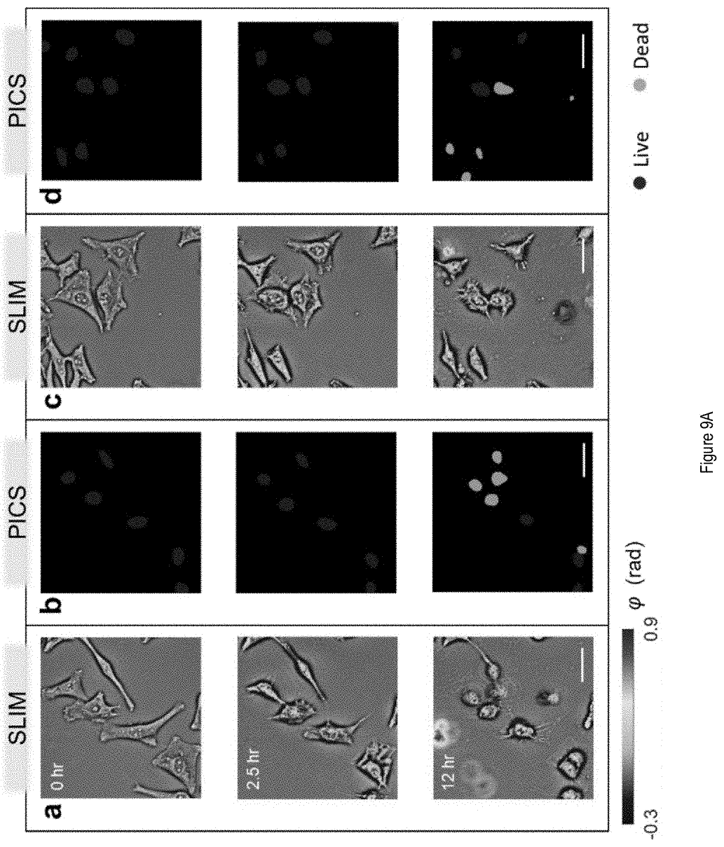

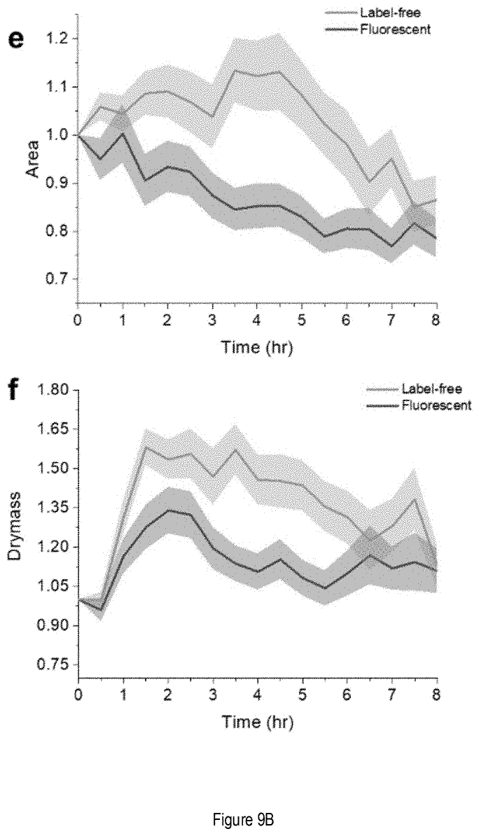

In FIG. 5A, panels a and c show SLIM images of HeLa cells with and without fluorescent reagents at t=0, 2.5, and 12 hours, respectively, whereas the resulting PICS predictions are shown in panel b and panel b. A time-lapse SLIM measurement, PICS prediction, and standard live-dead assay may be shown based on fluorescent measurements. HeLa cells may be shown without reagents. As expected, the PICS method depicts the transition from live to dead state. In addition, the visual comparison from FIG. 9A suggests that HeLa cells with viability stains in the media appear smaller in size, and more rapidly entering the injured state, as compared to their label-free counterparts. Using TrackMate, an imageJ plugin, it was able to extract the trajectory of individual cells and track their morphology over time. As a result, the cell nucleus, area, and dry mass at each moment in time can be obtained by integrating the pixel value over the segmented area in the PICS prediction and SLIM image, respectively. 57 labeled and 34 unlabeled HeLa cells may be tracked. In FIG. 9B, panels e-f show the area and dry mass change (mean±standard error), where the values are normalized with respect to the one at t=0. The results of tracking agree with the physiological description, and are consistent with previous reported experimental validations. However, the short swelling time in the reagent-treated cells suggest the toxicity of the chemical compounds would potentially accelerate the pace of cell death. Running two sample t-tests, a significant difference may be found in cell nuclear areas between the labelled and unlabeled cells, during the interval t=2 and t=7 hours (p<0.05). Similarly, cell dry mass showed significant differences between the two groups during the time interval t=2 and t=5 hours (p<0.05). This study may focus on optimizing the PICS performance in classifying live/dead markers at the cellular level. At the pixel level, the trained network can reveal the cell shape change, but its performance in capturing the nucleus shape and area is limited, which makes the current approach subject to segmentation error. This may be largely due to the low contrast at between nucleus boundary and cytoplasm in injured cells.

FIGS. 9A and 9B show viability of HeLa cells with and without reagent stains. In FIG. 9A, a. SLIM images of cells recorded at 0, 2.5 and 12 hours after staining. b. The PICS prediction associated with the frames in a. c. SLIM images of unstained HeLa cells measured at same time points as a. d. The corresponding PICS prediction associated with the frames in c. In FIG. 9B, e. Relative cell nuclear area change of tracked cells. The shaded region represents the standard error. f. Relative cell nucleus dry mass change. The shaded region represents of the standard error.

Although the effect of the fluorescent dye itself to the optical properties of the cell at the imaging wavelength is negligible, training on images of tagged cells may potentially alter the cell death mechanism and introduce bias when optimizing the E-U-Net. In order to investigate this potential concern, a set of experiments may be performed where the unlabeled cells were imaged first by SLIM, then tagged and imaged by fluorescence for ground truth. The performance of PICS in this case was consistent with the results showed in FIGS. 7 and 8, where SLIM was applied to tagged cells. The data indicated that the live and dead cells were classified with 99% and 97% sensitivity, respectively, suggesting that the proposed live-dead assay method can be used efficiently on cells that were never labeled. Of course, SLIM imaging of already stained cells, followed by fluorescence imaging, is a more practical workflow, as the input—ground truth image pairs can be collected continuously. On the other hand, training on unlabeled cells may achieve the true label-free assay which is most valuable in applications.

This embodiment demonstrated PICS as a method for high-speed, label-free, unbiased viability assessment of adherent cells. This may be the first method to provide live-dead information on unlabeled cells. This approach utilizes quantitative phase imaging to record high-resolution morphological structure of unstained cells, combined with deep learning techniques to extract intrinsic viability markers. Tested on HeLa and CHO adherent cultures, the optimized E-U-Net method reports outstanding accuracy of 96.7% and 94.9% in segmenting the cell nuclei and classifying their viability state. The E-U-Net accuracy may be compared with the outcomes from other networks or training strategies. By integrating the trained network on NVIDIA graphic processing units, the proposed label-free method enables real-time acquisition and viability prediction. One SLIM measurement and deep learning prediction takes ˜100 ms, which is approximately 8 times faster than the acquisition time required for fluorescence imaging with the same camera. Of course, the cell staining process itself takes time, approximately 15 minutes. The real-time in situ feedback is particularly useful in investigating viability state and growth kinetics in cells, bacteria, and samples in vivo over extended periods of time. In addition, results suggest that PICS rules out the adverse effect on cell function caused by the exogenous staining, which is beneficial for the unbiased assessment of cellular activity over long periods of time (e.g., many days). Of course, this approach can be applied to other cell types and cell death mechanisms.

Prior studies typically tracked QPI parameters associated with individual cells over time to identify morphological features correlated with cell death. In contrast, this approach provides real-time classification of cells based on single frames, which is a much more challenging and rewarding task. Compared to these previous studies, the PICS method avoids intermediate steps of feature extraction, manual annotation, and separate algorithms for training and cell classification. A single DNN architecture is employed with direct QPI measurement as input, and the prediction accuracy is significantly improved over the previously reported data. The labels outputted by the network can be used to create binary masks, which in turn yield dry mass information from the input data. The accuracy of these measurements depends on the segmentation process. Thus, it may be anticipated that future studies will optimize further the segmentation algorithms to yield high-accuracy dry mass measurements over long periods of time.

Label-free imaging methods are valuable for studying biological samples without destructive fixation or staining. For example, by employing infrared spectroscopy, the bond-selective transient phase imaging measures molecular information associated with lipid droplet and nucleic acids. In addition, harmonic optical tomography can be integrated into an existing QPI system to report specifically on non-centrosymmetric structures. These additional chemical signatures would potentially enhance the effective learning and produce more biophysical information. It may be anticipated that the PICS method will provide high-throughput cell screening for a variety of applications, ranging from basic research to therapeutic development and protein production in cell reactors. Because SLIM can be implemented as an upgrade module onto an existing microscope and integrates seamlessly with fluorescence, one can implement this label-free viability assay with ease.

FIG. 10 shows an evaluation result of the E-U-Net performance. An object-based accuracy metric is used to estimate the deep learning prediction by comparing the dominant semantic label of HeLa cell nuclei with the ground truth. The entries of the confusion are normalized with respect to number of cells in each class.

FIG. 11 shows another evaluation result of the E-U-Net performance on CHO with apoptosis reagents. The trained network yields high confidence in identifying live or apoptotic CHO cells. The entries of the confusion are normalized with respect to number of cells in each class.

HeLa cell preparation. HeLa cervical cancer cells (ATCC CCL-2™) and Chinese hamster ovary (CHO-K1 ATCC CCL-61™) cells were purchased from ATCC and kept frozen in liquid nitrogen. Prior to the experiments, the cells were thawed and cultured into T75 flask in Dulbecco's Modified Eagle Medium (DMEM with low glucose) containing 10% fetal bovine serum (FBS) and incubated in 37° C. with 5% CO2. As the cells reach 70% confluence, the flask was washed thoroughly with phosphate-buffered saline (PBS) and trypsinized with 3 mL of 0.25% (w/v) Trypsin EDTA for three minutes. When the cell starts to detach, the cells were suspended in 5 mL DMEM and passaged onto a glass bottom 6 well plate to grow. To evaluate the effect of confluency on PICS performance, CHO cells were plated in three different confluency levels: high (60000 cells), medium (30000 cells) and low (15000 cells). HeLa and CHO cells were then imaged after two days.

SLIM imaging. The SLIM optical setup in shown in FIG. 5A. In brief, the microscope is built upon an inverted phase contrast microscope using a SLIM module (CellVista SLIM Pro; Phi Optics) attached to the output port. Inside the module, a spatial light modulator (Meadowlark Optics) is placed at the system pupil plane via a Fourier transform lens to constantly modulate the phase delay between the scattered and incident light. By recording four intensity images with phase shifts of 0, π/2, π, and 3π/2, a quantitative phase map, φ, can be computed by combining the 4 acquired frames in real-time.

For both SLIM and fluorescence imaging, cultured cells were measured by a 40× objective, and the images were recorded by a CMOS camera (ORCA-Flash 4.0; Hamamatsu) with a pixel size of 6.5 μm. For each sample, a cellular region approximately 800×800 μm² was randomly selected to be measured by SLIM and fluorescence microscopy (NucBlue and NucGreen). The acquisition time of each SLIM and fluorescent measurement are 50 millisecond (ms) and 400 ms, respectively, and the scanning across all six-wells takes roughly 4.3 minutes, where the delay is caused by mechanical translation of the motorized stage. For deep learning training and predicting, the recorded SLIM images were downsampled by a factor of 2. This step saves computational cost and does not sacrifice information content. The acquisition of the fluorescence data is needed only for the training stage. For real-time interference, the acquisition is up to 15 frames per second for SLIM images, while the inference takes place in parallel.

E-U-Net architecture. The E-U-Net is a U-Net-like fully convolutional neural network that performs an efficient end-to-end mapping from SLIM images to the corresponding probability maps, from which the desired segmentation maps are determined by use of a softmax decision rule. Different from conventional U-Nets, the E-U-Net uses a more efficient network architecture, EfficientNet, for feature extraction in the encoding path. Here, EfficientNets refers to a family of deep convolutional neural networks that possess a powerful capacity of feature extraction but require much fewer network parameters compared to other state-of-the-art network architectures, such VGG-Net, ResNet, Mask R-CNN, etc. The EfficientNet family includes eight network architectures, EfficientNet-B0 to EfficientNetB7, with an increasing network complexity. EfficientNet-B3 and EfficientNet-B7 were selected for training E-U-Net on HeLa cell images and CHO cell images, respectively, considering they yields the most accurate segmentation performance on the validation set among all the eight EfficientNets. See FIGS. 6A and 6B for more details about the EfficientNet-B3 and EfficientNet-B7.

Loss function and network training. Given a set of B training images of M×N pixels and their corresponding ground truth semantic segmentation maps, loss function used for network training is defined as the combination of focal loss and dice loss:

$$\begin{matrix}{L}_{Focal \_ loss} = - \frac{1}{B} {\sum }_{i = 1}^{B} \frac{1}{MN} {\sum }_{x ∈ Ω} {[ 1 - {{y}_{i} ( x )}^{T} {p}_{i} ( x ) ]}^{γ} {{y}_{i} ( x )}^{T} {log}_{2} {p}_{i} ( x ) , \\ {L}_{Dice \_ loss} = 1 - \frac{1}{3} {\sum }_{c = 0}^{2} \frac{2 {TP}_{c}}{2 TP + {FP}_{c} + {FN}_{c}} \\ {L}_{combined} = α {L}_{Focal \_ loss} + β {L}_{Dice \_ loss}\end{matrix}$$

In the focal loss L_{Focal_loss}, Ω={(1,1), (1,2), . . . , (M,N)} is the set of spatial locations of all the pixels in a label map. yᵢ(x)∈{[1,0,0]^{T}, [0,1,0]^{T}, [0,0,1]^{T}} represents the ground truth label of the pixel x related to the i^{th} training sample, and the three one-hot vectors correspond to the live, dead and, background classes, respectively. Accordingly, the probability vector Pᵢ(x)∈□³ represents the corresponding predicted probabilities of belonging the three classes. [1−yᵢ(x)^{T}pᵢ(x)]^{γ} is a classification error-related weight that reduces the relative cross entropy yᵢ(x)^{T} log₂pᵢ(x) for well-classified pixels, putting more focus on hard, misclassified pixels. In this study, γ was set to be the default value of 2. As the dice loss L_{Dice_loss}, the TP_{c}, FP_{c}, and FN_{c} are the number of true positives, that of false positives, and that of false negatives, respectively, related to all pixels of viability class CE {0,1,2} in the B images. Here, c=0, 1, and 2 correspond to the live, dead and background classes, respectively. In the combined loss function, α,β∈{0,1} are two indicators that controls whether to use focal loss and dice loss in the training process, respectively. In this study, α, β was set to [1,0] and [1,1] for training the E-U-Nets on HeLa cell dataset and CHO cell dataset, respectively. The choices of [α,β] were determined by segmentation performance of the trained E-U-Net on the validation set. The E-U-Net was trained with randomly cropped patches of 512×512 pixels drawn from the training set by minimizing the loss function defined above with an Adam optimizer. In regard to Adam optimizer, the exponential decay rates for 1^{st} and 2^{nd} moment estimates were set to 0.9 and 0.999, respectively; a small constant ε for numerical stability was set to 10⁻⁷. The batch sizes were set to 14 and 4 for training the E-U-nets on the HeLa cell images and CHO cell images, respectively. The learning rate was initially set to 5×10⁻⁴. At the end of each epoch, the loss of a being-trained E-U-Net was computed on the whole validation set. When the validation loss did not decrease for 10 training epochs, the learning rate was multiplied by a factor of 0.8. This validation loss-aware learning rate decaying strategy benefits for mitigating the overfitting issue that commonly occurs in deep neural network training. Furthermore, data augmentation techniques, such as random cropping, flipping, shifting, and random noise and brightness adding etc., were employed to augment training samples on-the-fly for further reducing the overfitting risk. The E-U-Net was trained for 100 epochs. The parameter weights that yield the lowest validation loss were selected, and subsequently used for model testing and further model investigation.

The E-U-Net was implemented using the Python programming language with libraries including Python 3.6 and Tensorflow 1.14. The model training, validation and testing were performed on a NVIDIA Tesla V100 GPU of 32 GB VRAM.

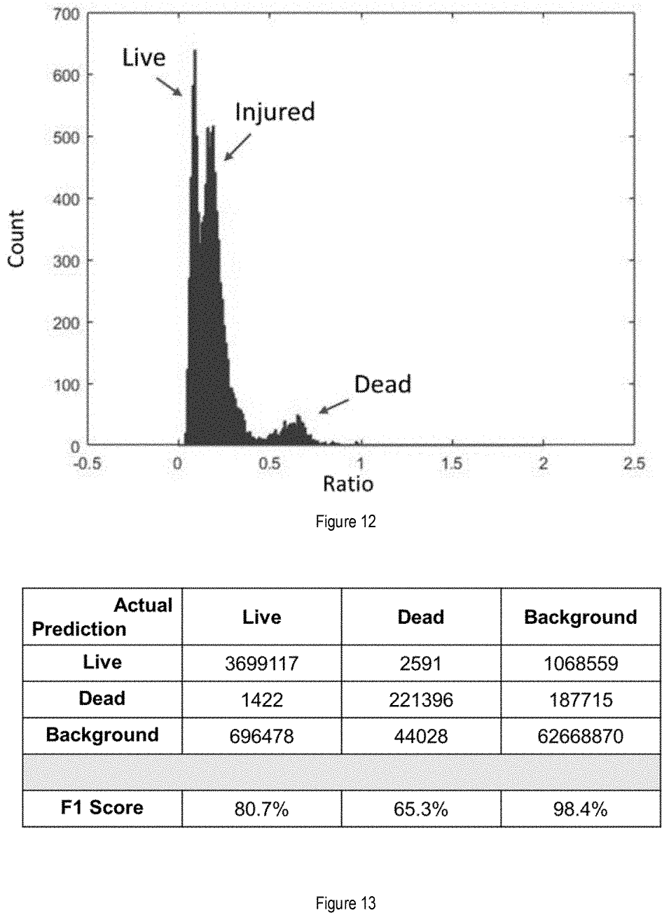

Semantic map generation: Semantic segmentation maps were generated in MATLAB with a customized script. First, for each NucBlue and NucGreen image pair, an adaptive thresholding was applied to separate the cell nucleus and background, where the segmented cell nuclei were obtained by computing the union of the binarized fluorescent image pair. The segmentation artifacts were removed by filtering out the tiny objects below the size of a typical nucleus. Next, using on the segmentation masks, the ratio between the NucGreen and NucBlue fluorescence signal was calculated. A histogram of the average ratio within the cell nucleus is plotted in FIG. 12, where three distinctive peaks were observed corresponding to the live, injured and dead cells. Because NucGreen/NucBlue reagent is only designed for live and dead classification, the histogram of injured cells is partially overlapped with the live cells. By selecting a threshold value that gives the lowest histogram count between dead and injured cells, label “live” was assigned to all live and injured cells, while the remaining cells as “dead”.

EfficientNet: The MBConvX is the principal module in an EfficientNet. It approximately factorizes a standard convolutional layer into a sequence of separable layers to shrink the number of parameters needed in a convolution operation while maintaining a comparable ability of feature extraction. The separable layers in a MBConvX module are shown in FIG. 6B. Here, MBConv1 (X=1) and MBConv6 (X=6) indicate that a ReLU layer and ReLU6 layer are employed in this module, respectively. ReLU6 is a modification of the rectified linear unit, where the activation is limited to a maximum size of 6. A MBConvX module in FIG. 6A may include a down-sampling layer, which can be inferred by the indicated feature map dimensions. The first MBConvX in each layer block does not contain a skip connection between its input and output (indicated as a dash line in panel c in FIG. 6B), since the input and output of that module have different sizes.

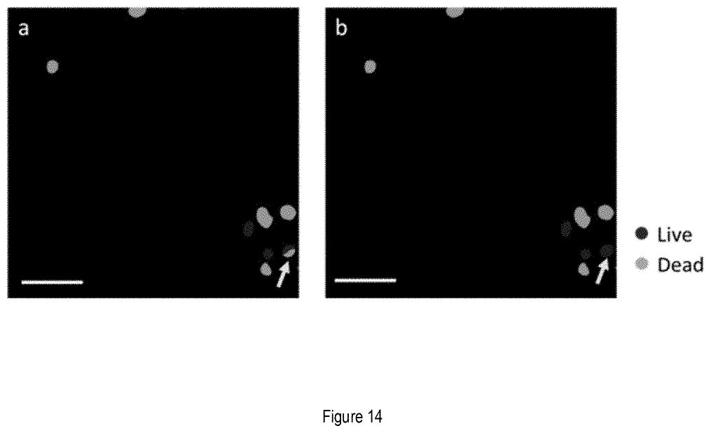

PICS evaluation at a cellular level: a U-Net based EfficientNet (E-U-Net) was implemented to extract markers associated with viable state of cells measured by SLIM. FIG. 13 shows the conventional confusion matrix and corresponding F1 score evaluated on pixels in testing images. In FIG. 14, panel a shows a represented raw E-U-Net output image. As indicated by the yellow arrow, there exist cases where a segmented cell may have multiple semantic labels. The conventional deep learning evaluation method only focuses on assessing pixel-wise segmentation accuracy, which overlooks some biologically relevant instances (the viable state of the entire cell). And this motives an adoption of an object-based evaluation that estimates the E-U-Net accuracy for individual cell.

FIG. 13 shows a pixel-wise evaluation of a trained E-U-Net. Due to the fact that the E-U-Net prediction assigns multiple labels to one cell nucleus, the pixel-wise classification was converted into cell-wise classification, which is more relevant biologically, as shown in FIG. 10.

First, dominant semantic label is used across a cellular region to denote the viable state for this cell (FIG. 14, panel b). This semantic label is compared with the same cell in ground truth image, this step is repeated across all testing images, and the cell-wise evaluation is obtained as shown in FIG. 10.

In FIG. 14, a. Output of E-U-Net on a representative testing image. The network assigns semantic labels to each pixel, and thus for some cells, more than one semantic label can be observed within the cell body. b. the dominant semantic label is used to indicate the viability state of a cell, and then the performance of training is evaluated at a cellular level, referred to the cell-wise evaluation. The images are randomly selected from a combined dataset across 4 imaging experiments. Scale bars represent 50 μm in space.

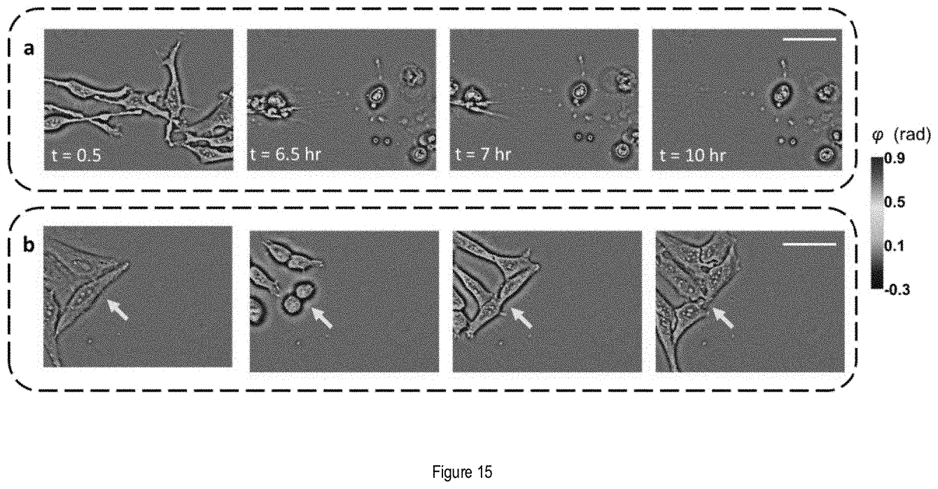

PICS on CHO cells and Evaluate the effect of lytic cell death: Before performing experiments on CHO cells, a preliminary study was conducted, as follows. Live cell cultures were prepared and split into the two groups. 1 μM of staurosporine was added into the medium of the experimental group, whereas the others were kept intact as control. Both control and experimental cells were measured with SLIM for 10 hours under regular incubation condition (37° C. and 5% concentration of CO₂). FIG. 15 shows the QPI images of experimental and control cells measured at t=0.5, 6.5, 7 and 10 hours, respectively. Throughout the time-course, the untreated cells remained attached to the petri-dish. Moreover, as indicated by the yellow arrows, the control cells divided at t=6.5 hr. In contrast, cells treated with staurosporine presented drastically different characteristics, where the cell volume decreased, and membrane ruptured or became detached. This preliminary result suggests that, under the regular incubation condition, the cells did not suffer from lytic cell death.

In FIG. 15 shows time-lapse SLIM recording of CHO cells with (a) and without (b) staurosporine that introduces cell apoptosis, under regular incubation condition. For the control group, the cells continued growing and dividing without signs of cell death, which ruled out the existence of lytic cell death. The images are selected from 1 experiments, and the results are consistent across 27 measured field of views (FOV). Scale bar represents 50 μm in space.

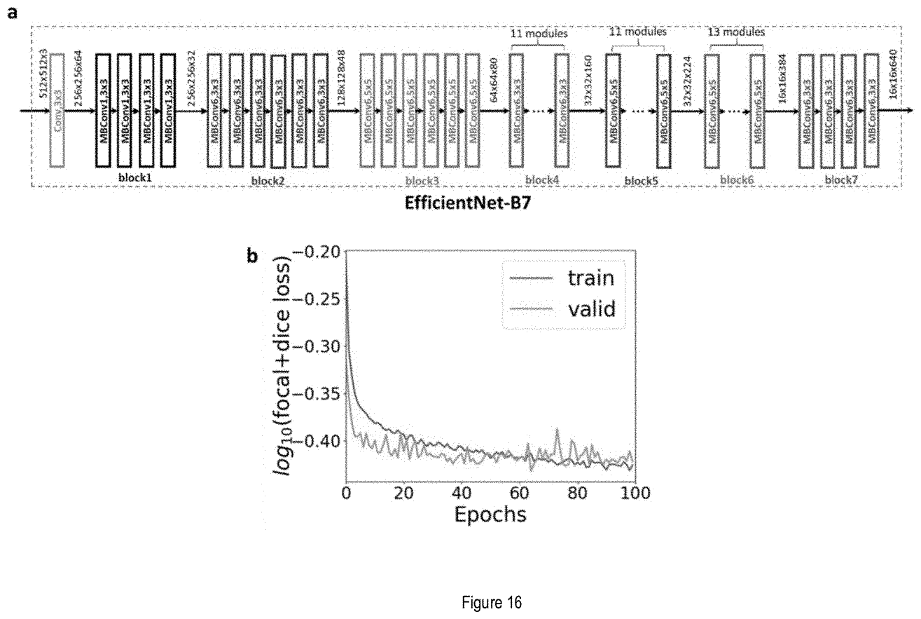

PICS training and testing on CHO cell images: After validation the efficacy of staurosporine on introducing apoptotic cell death, images on CHO cells were acquired and the dataset for PICS training were generated. The training was conducted on E-U-Net (EfficientNet-B7), whose network architecture, and its training/validation loss are shown in FIG. 16.

FIG. 16 shows CHO cells viability training with EfficientNet-B7. a. The network architecture of EfficientNet-B7. b. Training and validation focal losses vs number of epochs plotted in the log scale.

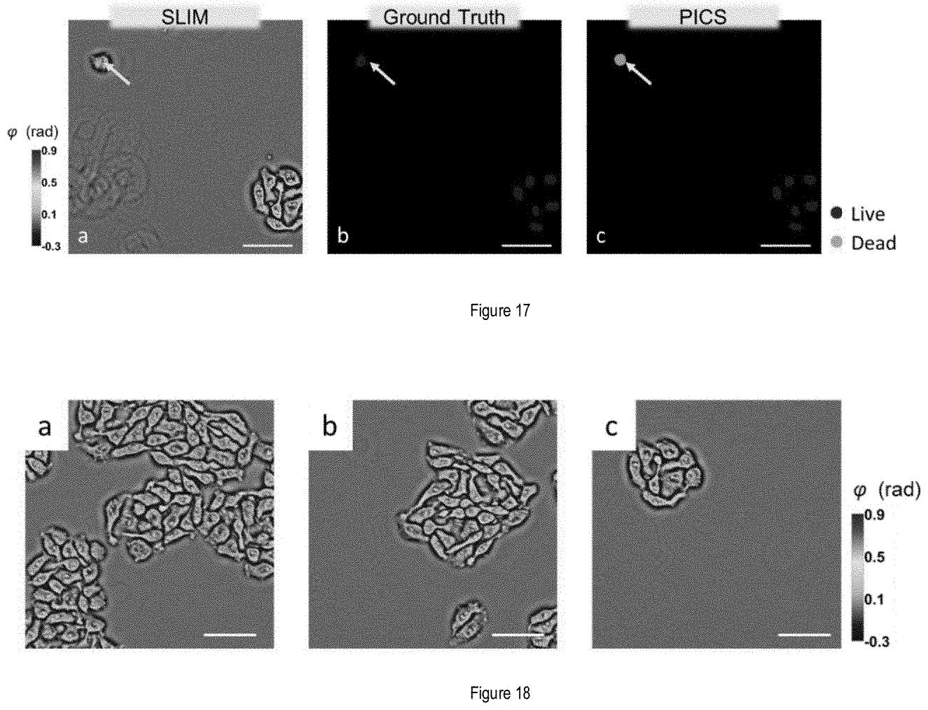

The difference between the ground truth and machine learning prediction in the testing dataset was visually inspected. First, there are prediction errors due to cells located at the boundary of the FOV, as explained in the previous comments. In addition, there are rare cases where live CHO cells were mistakenly labeled as dead (see FIG. 17 for an illustration of CHO cells with staurosporine administration at t=0.5 hour). In SLIM, these cells present features of abnormal cell shapes and decreased phase values, but severe membrane rupture was not observed. Previous studies suggested that these morphological features are early indicators of cell death, but it was identified as live using traditional fluorometric evaluation.

In FIG. 17, cells with irregular shapes but no severe membrane rupture are subjected to erroneous classification. a. Input SLIM image. b. Ground truth. c. PICS output based on input in a. The images are randomly selected from a combined dataset across 4 imaging experiments. Scale bar represents 50 μm in space.

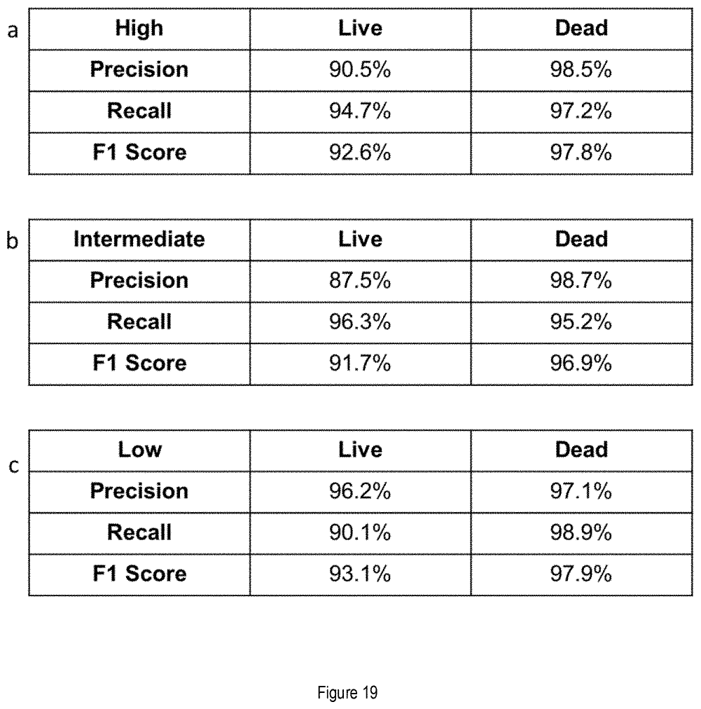

PICS performance on cells under different confluence: live CHO cell culture was prepared in a 6-well plate at three confluence levels, staurosporine solution was added into the culture medium to introduce apoptosis. FIG. 18 shows SLIM image of high, intermediate, and low confluence CHO cells measured at t=0. Although, aggregating into clusters, the cell shape and boundary can be easily identified. All SLIM images were combined for training and validation. In testing, the PICS performance vs. cell confluence was estimated, and the results are summarized in FIG. 19, showing PICS performance vs. CHO cell confluence.

In FIG. 18, SLIM images of high (a), intermediate (b), and low (c) confluence CHO cells. The images are randomly selected from a combined dataset across 4 imaging experiments. Scale bar represents 50 μm in space.

Training on unlabeled cell SLIM images: During the data acquisition, FL viability reagents were added at the beginning, and this allows monitoring the viable state changes of the individual cells over time. However, such data acquisition strategy can, in principle, introduce bias when optimizing the E-U-Net. This effect can be ruled out by collecting label-free images first, followed by exogenous staining and fluorescent imaging to obtain the ground truth, at the cost of increased efforts in staining, selecting FOV and re-focusing.

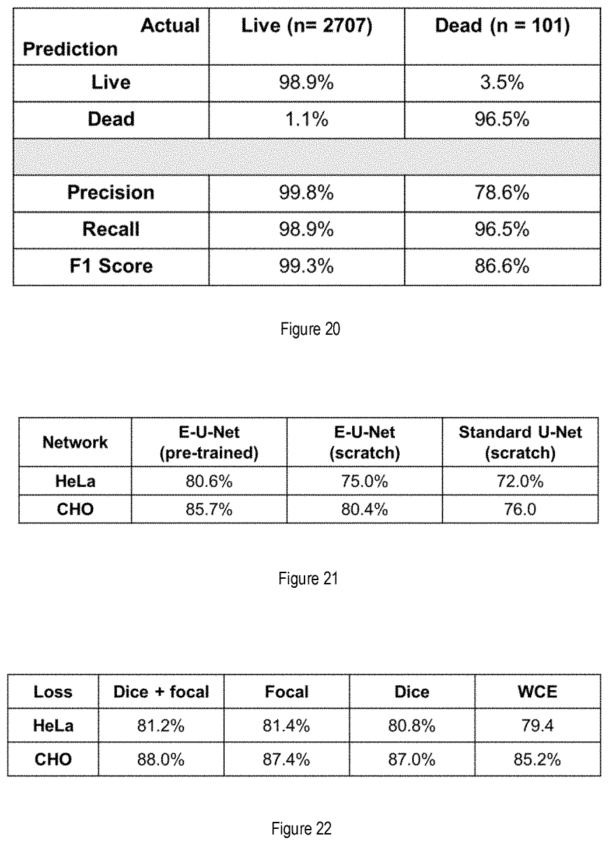

To study this potential effect, a control experiment described as follows was performed. Live CHO cells were prepared and passaged onto two glass-bottom 6-well plates. 1 μM of staurosporine was added into each well to introduce apoptosis. At t=0, cells in one well were imaged by SLIM, followed by reagents staining and fluorescence imaging. After 60 minutes, this step was repeated, but the cells in the other well was measured. Throughout the experiment, the cells were maintained in 37° C. and 5% concentration of CO₂. In this way, cells in each well were only measured once, and a dataset of unlabeled QPI images was obtained that resemble the structure of a testing dataset used in this study. The experiment was repeated 4 times, resulting in a total of 2400 SLIM and fluorescent pairs, on which PICS training and testing were performed. FIG. 20 shows the PICS performance on this new dataset, where live and dead cells were classified with 99% and 97% sensitivity, respectively. Thus, PICS optimization on cells without fluorescent stains does not compromise the prediction accuracy, which makes the proposed live-dead assay method robust for a variety of experiment settings.

FIG. 20 shows evaluation of the PICS performance on truly unlabeled CHO cells with apoptosis reagents.

Comparison of PICS performance under various training strategies: cell viability prediction performance under various network architecture settings were compared. three network settings were compared: 1) an E-U-net trained by use of a pre-trained EfficientNet; 2) an E-U-net trained from scratch; and 3) a standard U-net trained from scratch. In these additional experiments, the U-net architecture employed was a standard U-net, with the exception that batch normalization layers were placed after each convolutional layer to facilitate the network training. EfficientNet-B0 was employed in the E-U-nets to make sure that the network size of E-U-net (7.8 million of parameters) approximately matched that of a standard U-net (7.85 million of parameters). A combined loss that comprised focal and dice losses (denoted as dice+focal loss) was used for network training. Other training settings were consistent with how the E-U-net was trained, as described in the manuscript. After the networks were trained with training and validation data from HeLa cell datasets and CHO cell datasets, they were tested on the testing data from the two datasets, respectively. The average pixel-wise F1 scores over the live, dead and background classes were computed to evaluate the performance of the trained networks, as shown in FIG. 21. It can be observed that, on both the two testing datasets, the average F1 scores corresponding to an E-U-net are much higher than those corresponding to a standard U-net when both of them were trained from scratch. Furthermore, as expected, an E-U-net trained with a pre-trained EfficientNet achieves a better performance than the one trained from scratch. These results demonstrate the effectiveness of the E-U-net architecture and the transfer learning techniques in training a deep neural network for pixel-wise cell viability prediction.

FIG. 21 shows average F1 scores related to E-U-nets trained with a pre-trained EfficientNet-B0, E-U-nets trained from scratch, and standard U-nets trained from scratch, respectively.

In addition, the average pixel-wise F1 scores corresponding to E-U-nets trained with various loss functions were compared, including a dice+focal loss, a standard focal loss, a standard dice loss, and a weighted cross entropy (WCE) loss. To be consistent with the network settings in the manuscript, a pre-trained EfficientNet-B3 and a pre-trained EfficientNet-B7 were employed for training the E-U-nets on the HeLa cell dataset and CHO cell datasets, respectively. The class weights related to live, dead, and background classes in the weighted cross entropy loss were set to [0.17, 2.82, 0.012] and [2.32, 0.654, 0.027] for the network training on the HeLa cell dataset and CHO cell datasets, respectively. In each of the weight cross entropy losses, the average of weights over the three classes is 1, and the weights related to each class were inversely proportional to the percentages of pixels from each class in the HeLa cell and CHO cell training datasets: [6.7%, 0.4%, 92.9%] and [1.1%, 3.9%, 95%], respectively. Other network training settings were consistent with how the E-U-net was trained as described in the manuscript. The trained networks were then evaluated on the testing HeLa cell dataset containing 100 images and testing CHO cell dataset containing 288 images, respectively. The average pixel-wise F1 scores were computed over all pixels in the two testing sets as shown in FIG. 22. It can be observed that, on both the two datasets, E-U-nets trained with a dice+focal loss produced higher average pixel-wise F1 scores than those trained with a dice loss or a WCE loss.

FIG. 22 shows average F1 scores related to E-U-nets trained with various loss functions.

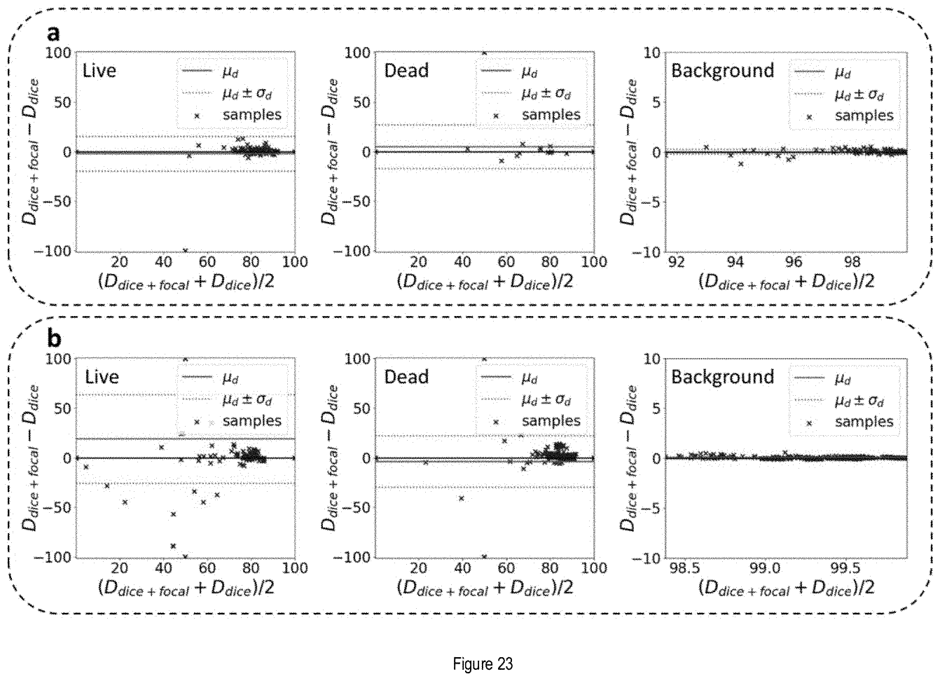

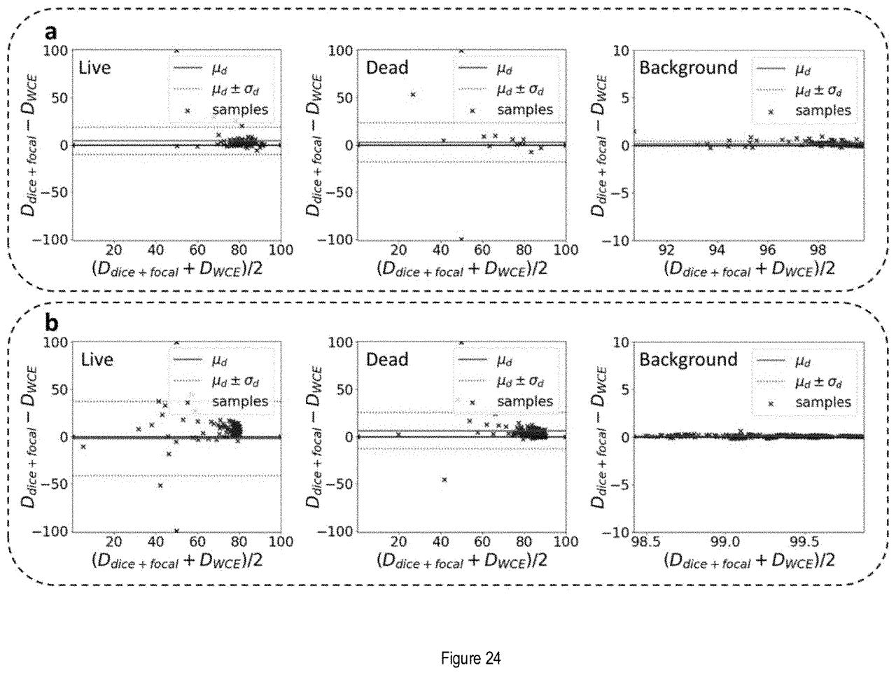

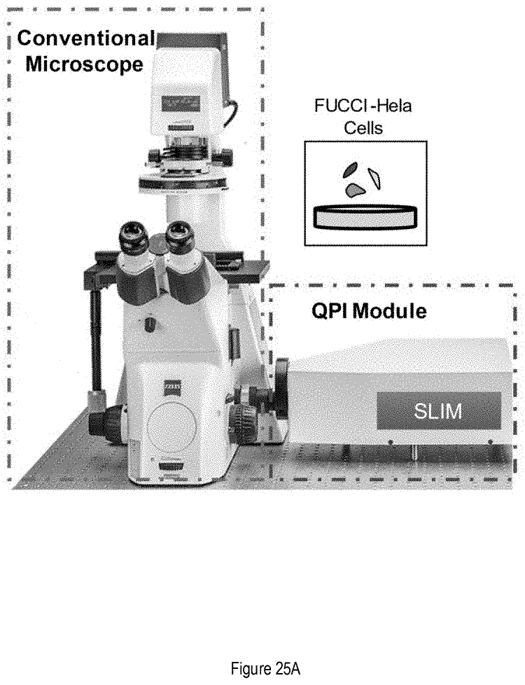

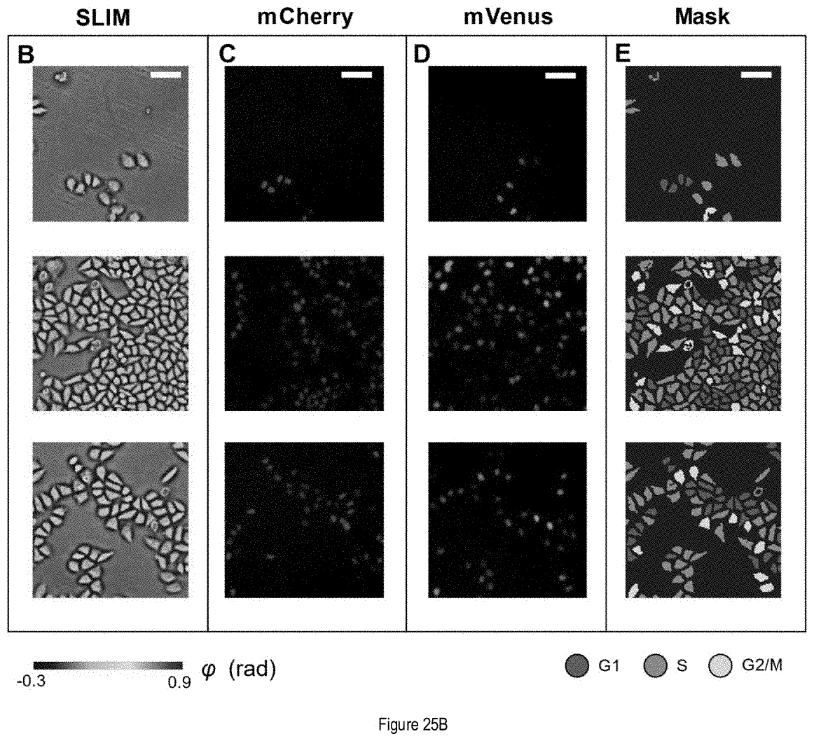

E-U-nets trained with a dice+focal loss to those trained with a dice loss or a WCE loss were further compared by investigating their agreements on the dice coefficients of each class related to the predictions for each image sample in the two testing datasets. Here, D_{dice+focal}, D_{dice}, and D_{WCE} are denoted as the dice coefficients produced by E-U-nets trained with a dice+focal loss, a dice loss and a weighted cross entropy loss, respectively. Bland-Altman plots were employed to analyze the agreement between D_{dice+focal} and D_{dice} and that between D_{dice+focal} and D_{WCE} on testing dataset of HeLa and that of CHO, respectively. Here, a Bland-Altman plot of two paired dice coefficients (i.e. D_{dice+focal} VS. D_{dice}) produces a scatter plot x-y, in which the y axis (vertical axis) represents the difference between the two paired dice coefficients (i.e. D_{dice+focal}−D_{dice}) and the x axis (horizontal axis) shows the average of the two dice coefficients (i.e. (D_{dice+focal}+D_{dice})/2). μ_{d} and σ_{d} represent the mean and standard deviation of the differences of the paired dice coefficients over the image samples in a specific testing dataset. The results corresponding to D_{dice+focal} vs. D_{dice} and D_{dice+focal} vs. D_{WCE} are reported in FIG. 23 and FIG. 24, respectively. In each figure, the subplots from left to right show the Bland-Altman plots related to the predictions for live, dead, and background classes, respectively. It can be observed that, for predicting live and dead pixels, both the D_{dice+focal}>D_{dice} (or D_{dice}+_{focal}−D_{dice}>0) and D_{dice}+_{focal}>D_{WCE} (or D_{dice}+_{focal}−D_{WCE}>0) hold at the majority of the image samples in the two datasets, though for the background prediction, D_{dice+focal} is comparable to D_{dice} and D_{WCE}. These results suggest that compared to a dice or WCE loss, a focal+dice loss can improve the performance of predicting live and dead pixels for the majority of testing images from both the two datasets.

FIG. 23 shows D_{dice+focal} vs. D_{dice} on testing dataset of HeLa (a) and CHO (b), where μ_{d} and σ_{d} represent the mean and standard deviation of D_{dice+focal}−D_{dice}. FIG. 24 shows D_{dice+focal} vs. D_{WCE} on testing dataset of HeLa (a) and CHO (b), where μ_{d} and σ_{d} represent the mean and standard deviation of D_{dice+focal}−D_{WCE}.

### Embodiment: A Cell Cycle Stage Classification Using Phase Imaging with Computational Specificity

Traditional methods for cell cycle stage classification rely heavily on fluorescence microscopy to monitor nuclear dynamics. These methods inevitably face the typical phototoxicity and photobleaching limitations of fluorescence imaging. Here, the present disclosure describes a cell cycle detection workflow using the principle of phase imaging with computational specificity (PICS). The method uses neural networks to extract cell cycle-dependent features from quantitative phase imaging (QPI) measurements directly. Results indicate that this approach attains very good accuracy in classifying live cells into G1, S, and G2/M stages, respectively. The present disclosure also demonstrates that the method can be applied to study single-cell dynamics within the cell cycle as well as cell population distribution across different stages of the cell cycle. The method may become a nondestructive tool to analyze cell cycle progression in fields ranging from cell biology to biopharma applications.

The cell cycle is an orchestrated process that leads to genetic replication and cellular division. This precise, periodic progression is crucial to a variety of processes, such as, cell differentiation, organogenesis, senescence, and disease. Significantly, DNA damage can lead to cell cycle alteration and serious afflictions, including cancer. Conversely, understanding the cell cycle progression as part of the cellular response to DNA damage has emerged as an active field in cancer biology.

Morphologically, the cell cycle can be divided into interphase and mitosis. The interphase can further be divided into three stages: G1, S, and G2. Since the cells are preparing for DNA synthesis and mitosis during G1 and G2 respectively, these two stages are also referred to as the “gaps” of the cell cycle. During the S stage, the cells are synthesizing DNA, with the chromosome count increasing from 2N to 4N.

Traditional approaches for distinguishing different stages within the cell cycle rely on fluorescence microscopy to monitor the activity of proteins that are involved in DNA replication and repair, e.g., proliferating cell nuclear antigen (PCNA). A variety of signal processing techniques, including support vector machine (SVM), intensity histogram and intensity surface curvature, level-set segmentation, and k-nearest neighbor have been applied to fluorescence intensity images to perform classification. In recent years, with the rapid development of parallel-computing capability and deep learning algorithms, convolutional neural networks have also been applied to fluorescence images of single cells for cell cycle tracking. Since all these methods are based on fluorescence microscopy, they inevitably face the associated limitations, including photobleaching, chemical, and phototoxicity, weak fluorescent signals that require large exposures, as well as nonspecific binding. These constraints limit the applicability of fluorescence imaging to studying live cell cultures over large temporal scales.

Quantitative phase imaging (QPI) is a family of label-free imaging methods that has gained significant interest in recent years due to its applicability to both basic and clinical science. Since the QPI methods utilize the optical path length as intrinsic contrast, the imaging is non-invasive and, thus, allows for monitoring live samples over several days without concerns of degraded viability. As the refractive index is linearly proportional to the cell density, independent of the composition, QPI methods can be used to measure the non-aqueous content (dry mass) of the cellular culture. In the past two decades, QPI has also been implemented as a label-free tomography approach for measuring 3D cells and tissues. These QPI measurements directly yield biophysical parameters of interest in studying neuronal activity, quantifying sub-cellular contents, as well as monitoring cell growth along the cell cycle. Recently, with the parallel advancement in deep learning, convolutional neural networks were applied to QPI data as universal function approximators for various applications. It has been shown that deep learning can help computationally substitute chemical stains for cells and tissues, extract biomarkers of interest, enhance imaging quality, as well as solve inverse problems.

The present disclosure describes a new methodology for cell cycle detection that utilizes the principle of phase imaging with computational specificity (PICS). The approach combines spatial light interference microscopy (SLIM), a highly sensitive QPI method, with recently developed deep learning network architecture E-U-Net. The present disclosure demonstrates on live Hela cell cultures that the method classifies cell cycle stages solely using SLIM images as input. The signals from the fluorescent ubiquitination-based cell cycle indicator (FUCCI) were only used to generate ground truth annotations during the deep learning training stage. Unlike previous methods that perform single-cell classification based on bright-field and dark-field images from flow cytometry or phase images from ptychography, the method can classify all adherent cells in the field of view and perform longitudinal studies over many cell cycles. Evaluated on a test set consisting of 408 unseen SLIM images (over 10,000 cells), the method achieves F-1 scores over 0.75 for both the G1 and S stage. For the G2/M stage, a lower score of 0.6 was obtained, likely due to the round cells going out of focus in the M-stage. Using the classification data outputted by the method, binary maps that were used back into the QPI (input) images were created to measure single cell area, dry mass, and dry mass density for large cell populations in the three cell cycle stages. Because the SLIM imaging is nondestructive, all individual cells can be monitored over many cell cycles without loss of viability. The method can be extended to other QPI imaging modalities and different cell lines, even those of different morphology, after proper network retraining for high throughput and nondestructive cell cycle analysis, thus, eliminating the need for cell synchronization.

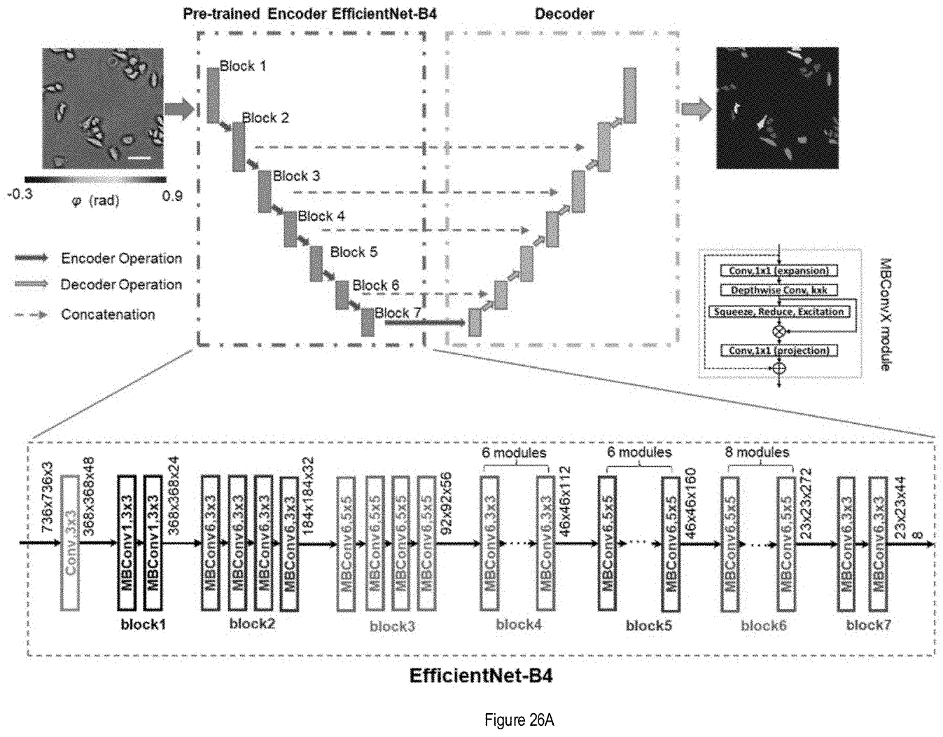

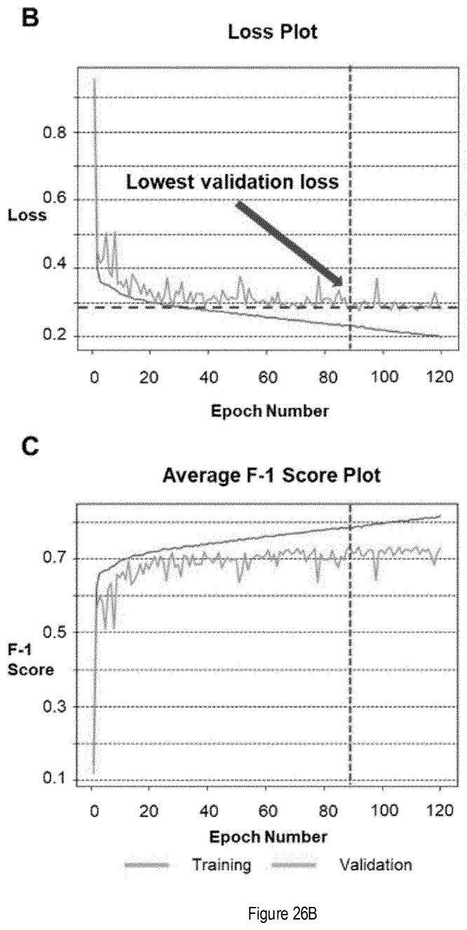

One exemplary experiment setup is illustrated in FIG. 25A. Spatial light interference microscopy (SLIM) was utilized to acquire the quantitative phase map of live HeLa cells prepared in six-well plates. By adding a QPI module to an existing phase contrast microscope, SLIM modulates the phase delay between the incident field and the scattered field, and an optical pathlength map is then extracted from four intensity images via phase-shifting interferometry. Due to the common-path design of the optical system, both the SLIM signals and epi-fluorescence signals of the same field of view (FOV) may be acquired using a shared camera. FIG. 25B shows the quantitative phase map of live HeLa cell cultures using SLIM.

To obtain an accurate classification between the three stages within one cell cycle interphase (G1, S, and G2), HeLa cells that were encoded with fluorescent ubiquitination-based cell cycle indicator (FUCCI) were used. FUCCI employs mCherry, an hCdt1-based probe, and mVenus, an hGem-based probe, to monitor proteins associated with the interphase. FUCCI transfected cells produce a sharp triple color-distinct separation of G1, S, and G2/M. FIG. 25B demonstrates the acquired mCherry signal and mVenus signal, respectively. the information from all three channels via adaptive thresholding were combined to generate a cell cycle stage mask (FIG. 25B). The procedure of sample preparation and mask generation is presented in detail in other paragraphs in the present disclosure.

FIGS. 25A and 25B show a schematic of the imaging system. In FIG. 25A, the SLIM module was connected to the side port of an existing phase contrast microscope. This setup allows us to take co-localized SLIM images and fluorescence images by switching between transmission and reflection illumination. In FIG. 25B, (B) Measurements of HeLa cells. (C) mCherry fluorescence signals. (D) mVenus fluorescence signals. (E) Cell cycle stage masks generated by using adaptive thresholding to combine information from all three channels. Scale bar is 100 μm.

With the SLIM images as input and the FUCCI cell masks as ground truth, the cell cycle detection problem may be formulated as a semantic segmentation task and trained a deep neural network to infer each pixel's category as one of the “G1”, “S”, “G2/M”, or background labels. the E-U-Net (FIG. 26A) is used as the network architecture. The E-U-Net architecture upgraded the classic U-Net by swapping its original encoder layers with a pre-trained EfficientNet. Since the EfficientNet was already trained on the massive ImageNet dataset, it provided more sophisticated initial weights than the randomly initialized layers from the scratch U-Net as in previous approaches. This transfer learning strategy enables the model to utilize “knowledge” of feature extraction learned from the ImageNet dataset, achieving faster convergence and better performance. Since EfficientNet was designed using a compound scaling coefficient, it is still relatively small in size. The trained network used EfficientNet-B4 as the encoder and contained 25 million trainable parameters in total.

The E-U-Net was trained with 2,046 pairs of SLIM images and ground truth masks for 120 epochs. The network was optimized by an Adam optimizer against the sum of the DICE loss and the categorical focal loss. After each epoch, the model's loss and overall F1-score were computed on both the training set and the validation set, which consists of 408 different image pairs (FIG. 26B). The weights of parameters that make the model achieve the lowest validation loss were selected and used for all verification and analysis. The training procedure is described in Methods.

FIGS. 26A and 26B shows a PICS training procedure. In FIG. 26A, a network architecture called the E-U-Net that replaces the encoder part of a standard U-Net with the pre-trained EfficientNet-B4 is used. Within the encoder path, the input images were downsampled 5 times through 7 blocks of encoder operations. Each encoder operation consists of multiple MBConvX modules that consist of convolutional layers, squeeze and excitation, and residual connections. The decoder path consists of concatenation, convolution and upsampling operations. In FIG. 26B, (B) The model loss values on the training dataset and the validation dataset after each epoch. The model checkpoint with the lowest validation loss was picked as the final model and used it for all analysis. (C) The model's average F-1 score on the training dataset and the validation dataset after each epoch.

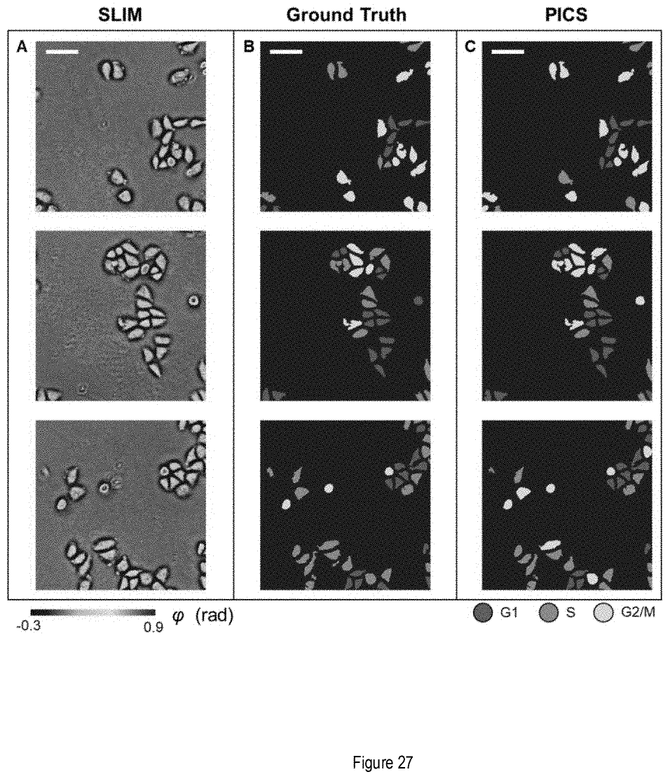

After training the model, its performance was evaluated on 408 unseen SLIM images from the test dataset. The test dataset was selected from wells that are different from the ones used for network training and validation during the experiment. FIG. 27, panel A shows randomly selected images from the test dataset. Panels B and C show the corresponding ground truth cell cycle masks and the PICS cell cycle masks, respectively. It can be seen that the trained model was able to identify the cell body accurately.

FIG. 27 shows PICS results on the test dataset. (A) SLIM images of Hela cells from the test dataset. (B) Ground truth cell cycle phase masks. (C) PICS-generated cell cycle phase masks. Scale bar is 100 μm.

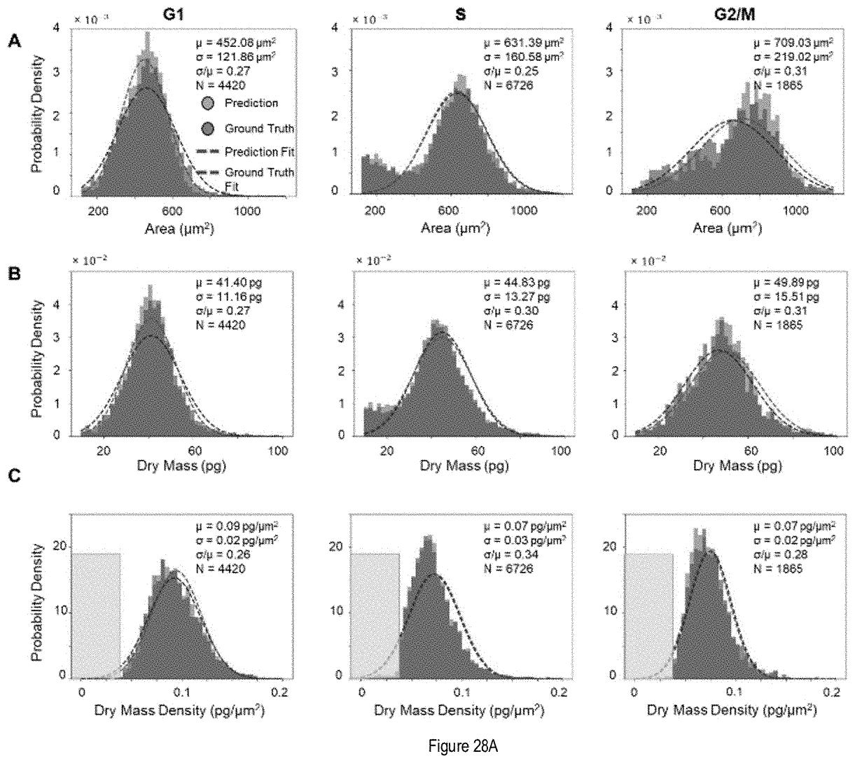

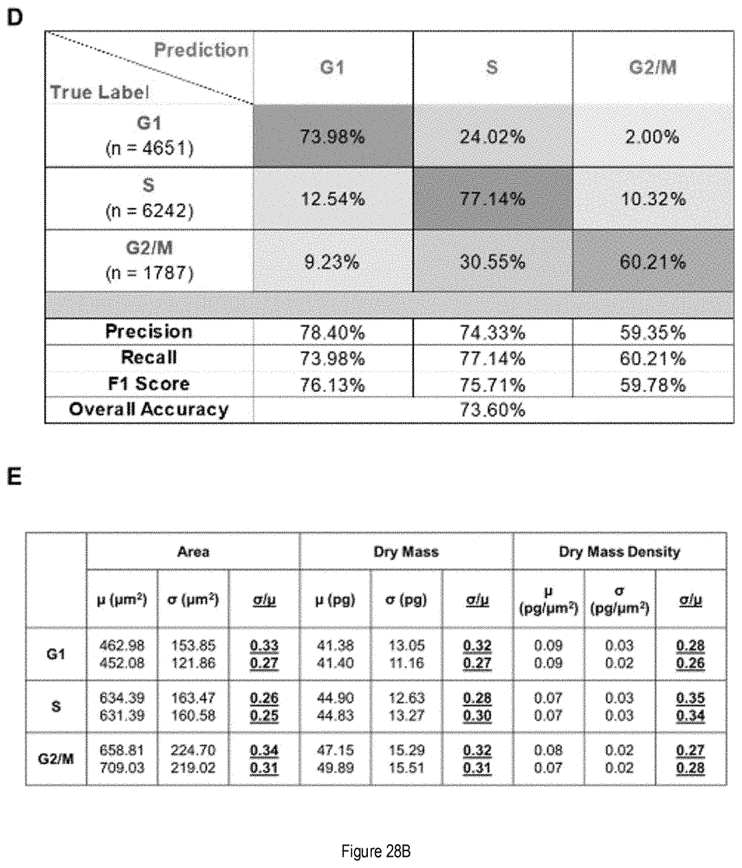

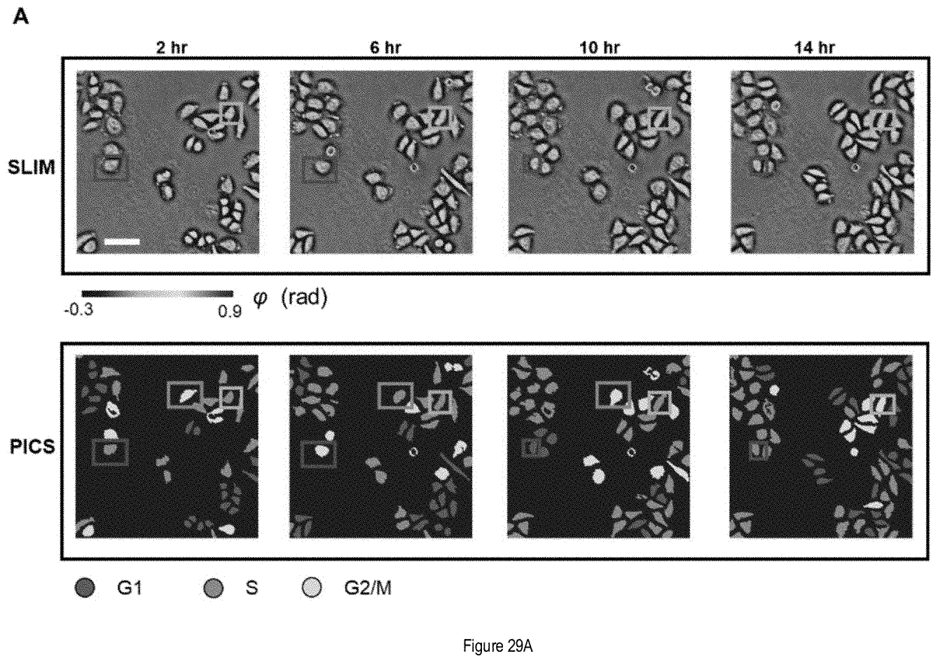

The raw performance of the PICS methods may be analyzed, with pixel-wise precision, recall, and F1-score for each class. However, these metrics did not reflect the performance in terms of the number of cells. Thus, a post-processing step on the inferred masks to enforce particle-wise consistency was performed. After this post-processing step, the model's performance was evaluated on the cellular level and produced the cell count-based results shown in FIGS. 28A and 28B. Panel A shows the histogram of cell body area for cells in different stages, derived from both the ground truth masks and the prediction masks. Panels B and C show similar histograms of cellular dry mass and dry mass density, respectively. The histograms indicated that there is a close overlap between the quantities derived from the ground truth masks and the prediction masks. The cell-wise precision, recall, and F-1 score for all three stages are shown in Panel D. Each entry is normalized with respect to the ground truth number of cells in that stage. The deep learning model achieved over 0.75 F-1 scores for both the G1 stage and the S stage, and a 0.6 F-1 score for the G2/M stage. The lower performance for the G2/M stage is likely due to the round cells going out of focus during mitosis. To better compare the performance of the PICS method with the previously reported works, two more confusion matrices may be produced by merging labels to quantify the accuracy of the method in classifying cells into [“G1/S”, “G2/M”] and [“G1”, “S/G2/M”]. For all the classification formulations, the overall accuracy was computed. Compared to the overall accuracy of 0.91¹³ from a method that used convolutional neural networks on fluorescence image pairs to classify single cells into “G1/S” or “G2”, the method achieved a comparable overall accuracy of 0.89. Compared to the F1-score of 0.94 and 0.88 for “G1” and “S/G2” respectively from a method that used convolution neural networks on fluorescence images, the method achieved a lower F-1 score for “G1” and a comparable F-1 score for “S/G2/M”. Comparted to the method that classifies single-cell images from flow cytometry, the method achieved a lower F-1 score for “G1” and “G2/M”. and a higher F-1 score for “S’.

The means and standard deviations of the best fit Gaussian were computed for the area, dry mass, and dry mass density distributions for populations of cells in each of the three stages: G1 (N=4,430 cells), S (N=6,726 cells), and G2/M (1,865 cells). The standard deviation divided by the mean, σ/μ, is a measure of the distribution spread. These values are indicated in each panel of FIG. 28A and summarized in FIG. 28B (the top parameter was from the ground truth population and the bottom parameter was from the PICS prediction population). The G1 phase is associated with distributions that are most similar to a Gaussian. It is interesting that the S-phase exhibits a bimodal distribution in both area and dry mass, indicating the presence of a subpopulation of smaller cells at the end of G1 phase. However, the dry mass density even for this bimodal population becomes monomodal, suggesting that the dry mass density is a more uniformly distributed parameter, independent of cell size and weight. Similarly, the G2/M area and dry mass distributions are skewed toward the origin, while the dry mass density appears to have a minimum value of ˜0.0375 pg/μm² (within the orange rectangles). Interestingly, early studies of fibroblast spreading also found that there is a minimum value for the dry mass density that cells seem to exhibit.

FIGS. 28A and 28B shows PICS performance on the test dataset. In FIG. 28A, (A-C) Histograms of cell area, dry mass and dry mass density for cells in G1, S, and G2/M, generated by the ground truth mask (in blue) and by PICS (in green). A Gaussian distribution (in blue) was fitted to the ground truth data and another Gaussian distribution (in red) was fitted to the PICS results. In FIG. 28B, (D) Confusion matrix for PICS inference on the test dataset. (E) Mean, standard deviation and their ratio (underlined for visibility) of cell area, dry mass and dry mass density obtained from the fitted Gaussian distributions. The top number is the fitted parameter on the ground truth population while the bottom number is fitted on the PICS prediction population.

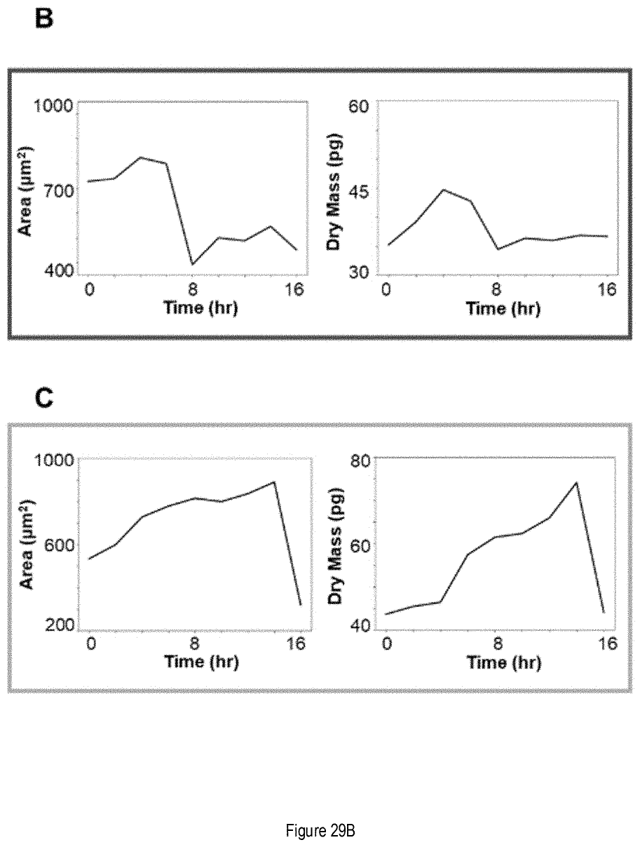

The PICS method may be applied to track the cell cycle transition of single cells, nondestructively. FIG. 29A shows the time-lapse SLIM measurements and PICS inference of HeLa cells. The time increment was roughly two hours between two measurements and the images at t=2, 6, 10, and 14 hours were displayed in FIG. 29A. The deep learning model has not seen any of these SLIM images during training. The comparison between the SLIM images and the PICS inference showed that the deep learning model produced accurate cell body masks and assigned viable cell cycle stages. FIG. 29B shows the results of manually tracking two cells in this field of view across 16 hours and using the PICS cell cycle masks to compute their cellular area and dry mass. Panel B demonstrates the cellular area and dry mass change for the cell marked by the red rectangle. An abrupt drop in both the area and dry mass around t=8 hours, at which point the mother cell divides into two daughter cells. The PICS cell cycle mask also captured this mitosis event as it progressed from the “G2/M” label to the “G1” label. A similar drop in Panel C after 14 hours due to mitosis of the cell marked by the orange rectangle. Panel C also shows that the cell continues growing before t=14 hours and the PICS cell cycle mask progressed from the “S” label to the “G2/M” label correspondingly. Note that this long-term imaging is only possible due to the nondestructive imaging allowed by SLIM. It is possible that the PICS inference will produce inaccurate stage label for some frames. For instance, PICS inferred label “G2/M” for the cell marked by the blue rectangle at t=2, 10 hours, but inferred label “S” for the same cell at t=6 hours. Such inconsistency can be manually corrected when the user made use of the time-lapse progression of the measurement as well as the cell morphology measurements from the SLIM image.

FIGS. 29A and 29B shows PICS on time lapse of FUCCI-Hela cells. In FIG. 29A, SLIM images and PICS inference of cells measured at 2, 6, 10, and 14 hours. The time interval between imaging is roughly 2 hours. two cells (marked in red and orange) were manually tracked. In FIG. 29B, (B) Cell area and dry mass change of the cell in the red rectangle, across 16 hours. These values were obtained via PICS inferred masks. An abrupt drop in cell dry mass and area as the cell divides after around 8 hours. (C) Cell area and dry mass change of the cell in orange rectangle, across 16 hours. The cell continues growing in the first 14 hours as it goes through G1, S, and G2 phase. It divides between hour 14 and hour 16, with an abrupt drop in its dry mass and cell area. Scale bar is 100 μm.

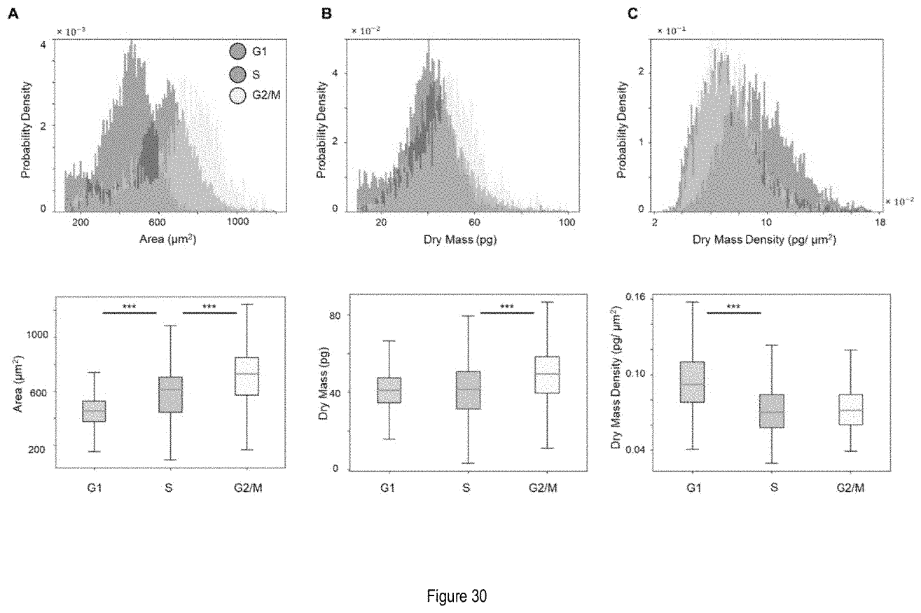

The present disclosure also demonstrates that the PICS method can be used to study the statistical distribution of cells across different stages within the interphase. The PICS inferred cell area distribution across G1, S, and G2/M is plotted in panel A in FIG. 30, whereby a clear shift between cellular area in these stages can be observed. Welch's t-test were performed on these three groups of data points. To avoid the impact on p-value due to the large sample size, 20% of all data points from each group were randomly sampled and the t-test were performed on these subsets instead. After sampling, there are 884 cells in G1, 1345 cells in S, and 373 cells in G2/M. The p-values are less than 10⁻³, indicating statistical significance. The same analysis was performed on the cell dry mass and cell dry mass density, as shown in panels B and C in FIG. 30. A clear distinction between cell dry mass in S and G2/M as well as between cell dry mass density in G1 and S. These results agree with the general expectation that cells are metabolically active and grow during G1 and G2. During S, the cells remain metabolically inactive and replicate their DNA. Since the DNA dry mass only accounts for a very small factor of the total cell dry mass, the distinction between G1 cell dry mass and S cell dry mass is less obvious than the distinction between S cell dry mass and G2/M cell dry mass. The cell dry mass density distribution agrees with previous findings.

FIG. 30 shows statistical analysis from PICS inference on the test dataset. (A) Histogram and box plot of cell area. The p-value returned from Welch's t-test indicated statistical significance. (B) Histogram and box plot of cell dry mass. The p-value returned from Welch's t-test indicated statistical significance. (C) Histogram and box plot of cell dry mass density. The p-value returned from Welch's t-test indicated statistical significance comparing cells in G1 and S. The box plot and Welch's t-test are computed on 20% of all data points in G1, S, and G2/M, randomly sampled. The sample size is 884 for G1, 1345 for S, and 373 for G2/M. Outliers are omitted from the box plot. (*** p<0.001).

The present disclosure describes a PICS-based cell cycle stage classification workflow for fast, label-free cell cycle analysis on adherent cell cultures and demonstrated it on the Hela cell line. The new method utilizes trained deep neural networks to infer an accurate cell cycle mask from a single SLIM image. The method can be applied to study single-cell growth within the cell cycle as well as compare the cellular parameter distributions between cells in different cell cycle phases.

Compared to many existing methods of cell cycle detection, this method yielded comparable accuracy for at least one stage in the cell cycle interphase. The errors in the PICS inference can be corrected when the time-lapse progression and QPI measurements of cell morphology were taken into consideration. Due to the difference in the underlying imaging modality and data analysis techniques, it is believed that this method has three main advantages. First, the method uses a SLIM module, which can be installed as an add-on component to a conventional phase contrast microscope. The user experience remains the same as using a commercial microscope. Significantly, due to the seamless integration with the fluorescence channel on the same field of view, the instrument can collect the ground truth data very easily, while the annotation is automatically performed via thresholding, rather than manually. Second, the method does not rely on fluorescence signals as input. On the contrary, the method is built upon the capability of neural networks to extract label-free cell cycle markers from the quantitative phase map. Thus, the method can be applied to live cell samples over long periods of time without concerns of photobleaching or degraded cell viability due to chemical toxicity, opening up new opportunities for longitudinal investigations. Third, the approach can be applied to large sample sizes consisting of entire fields of views and hundreds of cells. Since the task was formulated as semantic segmentation and the model was trained on a dataset containing images with various cell counts, the method worked with FOVs containing up to hundreds of cells. Also, since the U-Net style neural network is fully convolutional, the trained model can be applied to images with arbitrary size. Consequently, the method can directly extend to other cell datasets or experiments with different cell confluences, as long as the magnification and numerical aperture stay the same. Since the input imaging data is nondestructive, large cell populations may be imaged over many cell cycles and study cell cycle phase-specific parameters at the single cell scale. As an illustration of this capability, distributions of cell area, dry mass and dry mass density are measured for populations of thousands of cells in various stages of the cell cycle. The dry mass density distribution drops abruptly under a certain value for all cells, which indicates that live cells require a minimum dry mass density.

During the development of the method, standard protocols in the community were followed, such as preparing a diverse enough training dataset, properly splitting the training, validation and test dataset, and closely monitoring the model loss convergence to ensure that the model can generalize. Some studies showed that, with high-quality ground truth data, the deep learning-based methods applied to quantitative phase images are generalizable to predict cell viability and nuclear cytoplasmic ratio on multiple cell lines. Thus, although the method is only demonstrated on Hela cells due to the limited availability of cell lines engineered with FUCCI(CA)2, PICS-based instruments are well-suited for extending the method to different cell lines and imaging conditions with minimal effort to perform extra training. The typical training takes approximately 20 hours, while the inference is performed within 65 ms per frame. Thus, it is envisioned that the workflow is a valuable alternative to the existing methods for cell cycle stage classification and eliminates the need for cell synchronization.

FUCCI cell and HeLa cell preparation. HeLa/FUCCI(CA)2 cells were acquired from RIKEN cell bank and kept frozen in liquid nitrogen tank. Prior to the experiments, cells were thawed and cultured into T75 flasks in Dulbecco's Modified Eagle Medium (DMEM with low glucose) containing 10% fetal bovine serum (FBS) and incubated in 37° C. with 5% CO₂. When the cells reached 70% confluency, the flask was washed with phosphate-buffered saline (PBS) and trypsinized with 4 mL of 0.25% (w/v) Trypsin EDTA for four minutes. When the cells started to detach, they were suspended in 4 mL of DMEM and passaged onto a glass-bottom six-well plate. HeLa cells were then imaged after two days of growth.

SLIM imaging. The SLIM system architecture is shown in FIG. 25A. A SLIM module (CellVista SLIM Pro; Phi Optics) was attached to the output port of a phase contrast microscope. Inside the SLIM module, the spatial light modulator matched to the back focal plane of the objective controlled the phase delay between the incident field and the reference field. Four intensity images were recorded at phase shifts of 0, π/2, π, and 3π/2 and the quantitative phase map of the sample was reconstructed. Both the SLIM signal and the fluorescence signal were measured with a 10×/0.3NA objective. The camera used was Andor Zyla with a pixel size of 6.5 μm. The exposure time for SLIM channel and fluorescence channel was set to 25 ms and 500 ms, respectively. The scanning of the multi-well plate was performed automatically via a control software developed in-house. For each well, an area of 7.5×7.5 mm² was scanned, which took approximately 16 minutes for the SLIM and the fluorescence channels. The dataset used in this study were collected over 20 hours, with approximately 30 minutes interval between each round of scanning.

Cellular dry mass computation. The dry mass was recovered as

$$m ( x , y ) = \frac{λ}{2 πγ} ϕ ( x , y ) ,$$

using the same procedure outlined in previous works. λ=550 nm is the central wavelength; γ=0.2 ml/g is the specific refraction increment, corresponding to the average of reported values; and ϕ(x,y) is the measured phase. The above equation provides the dry mass density at each pixel, and the region of interest was integrated over to get the cellular dry mass.

Ground truth cell cycle mask generation. To prepare the ground truth cell cycle masks for training the deep learning models, information from the SLIM channel and the fluorescence channels were combined by applying adaptive thresholding. All the code may be implemented in Python, using the scikit-image library. The adaptive thresholding algorithm was firstly applied on the SLIM images to generate accurate cell body masks. Then the algorithm was applied on the mCherry fluorescence images and mVenus fluorescence images to get the nuclei masks that indicate the presence of the fluorescence signals. To ensure the quality of the generated masks, the adaptive thresholding algorithm was applied on a small subset of images with a range of possible window sizes. Then the quality of the generated masks was manually inspected and the best window size was selected to apply to the entire dataset. After getting these three masks (cell body mask, mCherry FL mask, and mVenus FL mask), the intersection was taken among them. Following the FUCCI color readout, a presence of mCherry signal alone indicates the cell is in G1 stage and a presence of mVenus signal alone indicates the cell is in S stage. The overlapping of both signals indicates the cell is in G2 or M stage. Since the cell mask is always larger than the nuclei mask, the entire cell area was filled in with the corresponding label. To do so, connected component analysis was performed on the cell body mask and the number of pixels marked by each fluorescence signal in each cell body was counted and the majority label was taken. The case of no fluorescence signal was handled by automatically labeling them as S because both fluorescence channels yield low-intensity signals only at the start of the S phase. Before using the mask for analysis, traditional computer vision operations were also performed, e.g., hole filling. on the generated masks to ensure the accuracy of computed dry mass and cell area.

Deep learning model development. The E-U-Net architecture was used to develop the deep learning model that can assign a cell cycle phase label to each pixel. The E-U-Net upgraded the classic U-Net architecture by swapping its encoder component with a pre-trained EfficientNet. Compared to previously reported transfer-learning strategies, e.g. utilizing a pre-trained ResNet for the encoder part, it may be believed that the E-U-Net architecture may be superior since the pre-trained EfficientNet attains higher performance on the benchmark dataset while remaining compact due to the compound scaling strategy.

The EfficientNet backbone ended up using for this project was EfficientNet-B4 (FIG. 26A). The entire E-U-Net-B4 model contains around 25 million trainable parameters, which is smaller compared to the number of parameters from the stock U-Net and other variations. The network was trained with 2046 image pairs in the training dataset and 408 image pairs in the validation dataset. Each image contains 736×736 pixels. The model was optimized using an Adam optimizer with default parameters against the sum of the DICE loss and the categorical focal loss. The DICE loss was designed to maximize the dice coefficient D between the ground truth label (gᵢ) and prediction label (pᵢ) at each pixel. It has been shown in previous works that DICE loss can help tackle class imbalance in the dataset. Besides DICE loss, the categorical focal loss FL(pₜ) was also utilized. The categorical focal loss extended the cross entropy loss by adding a modulating factor (1−pₜ)^{γ}. It helped the model to focus more on wrong inferences by preventing easily classified pixels dominating the gradient. The ratio between these two loss values was tuned and multiple training sessions were launched. In the end, the model trained against an equally weighted DICE loss and categorical focal loss gave the best results.

$$\begin{matrix}D = \frac{2 {\sum }_{i}^{N} {p}_{i} {ℊ}_{i}}{( {\sum }_{i}^{N} {p}_{i}^{2} + {\sum }_{i}^{N} {ℊ}_{i}^{2} )} \\ FL ( {p}_{t} ) = - {( 1 - {p}_{t} )}^{γ} log ( {p}_{t} )\end{matrix}$$

The model was trained for 120 epochs, taking over 18 hours on an Nvidia V-100 GPU. For learning rate scheduling, previous works was followed and learning rate warmup and cosine learning rate decay were implemented. During the first five epochs of training, the learning rate will increase linearly from 0 to 4×10⁻³. After that, the learning rate was decreased at each epoch following the cosine function. Based on experiments, relaxing the learning rate decay was ended up such that the learning rate in the final epoch will be half of the initial learning rate instead of zero. The model's loss value was plotted on both the training dataset and the validation dataset after each epoch (FIG. 26B) and picked the model checkpoint with the lowest validation loss as the final model to avoid overfitting. All the deep learning code was implemented using Python 3.8 and TensorFlow 2.3.

Post-processing. The performance of the trained E-U-Net was evaluated on an unseen test dataset and the precision, recall, and F-1 score were reported for each category: G1, S, G2/M, and background, respectively. The pixel-wise confusion matrix indicated the model achieved high performance in segmenting the cell bodies from the background. However, since this pixel-wise evaluation overlooked the biologically relevant instance, i.e., the number of cells in each cell cycle stage, an extra step of post-processing was performed to evaluate that.

Connected-component analysis was first performed on the raw model predictions. Within each connected component, a simple voting strategy was applied where the majority label will take over the entire cell. Enforcing particle-wise consistency, in this case, may be justified because it is impossible for a single cell to have two cell cycle stages at the same time and that the model is highly accurate in segmenting cell bodies, with over 0.96 precision and recall. The precision, recall, and F-1 score for each category on the cellular-level were then computed. For each particle in the ground truth, its centroid (or the median coordinates if the centroid falls out of the cell body) was used to determine if the predicted label matches the ground truth. The cellular-wise metrics were reported in FIG. 28B.

Before using the post-processed prediction masks to compute the area and dry mass of each cell, hole-filling was also performed as for the ground truth masks to ensure the values are accurate.

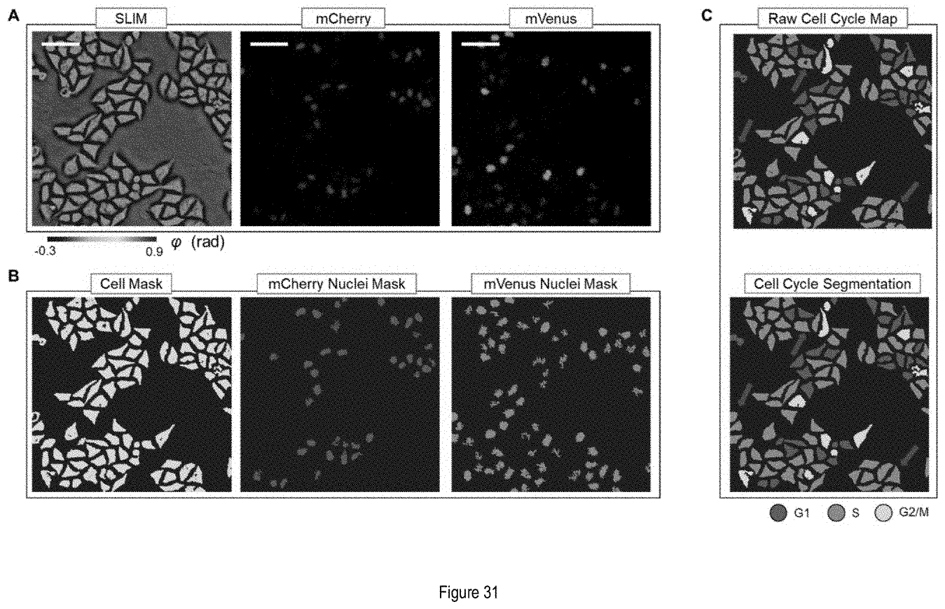

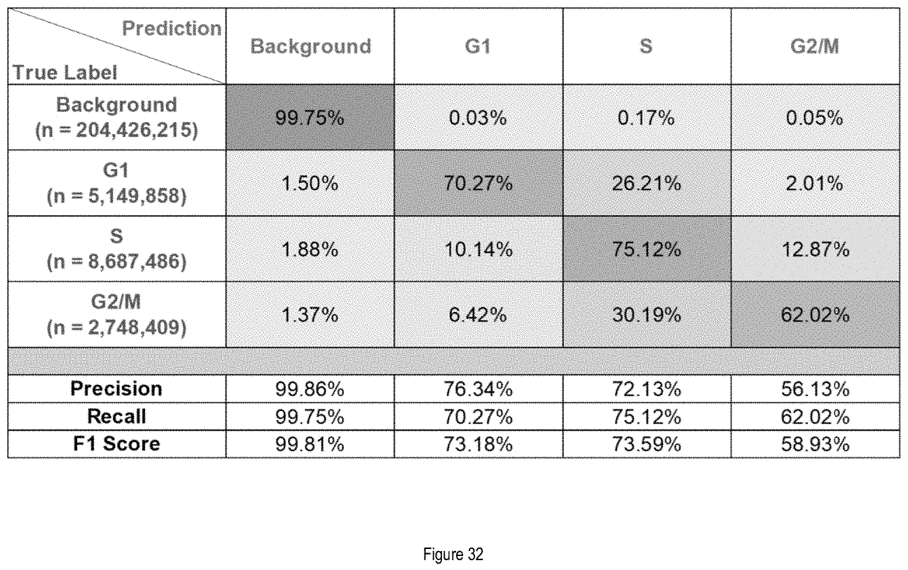

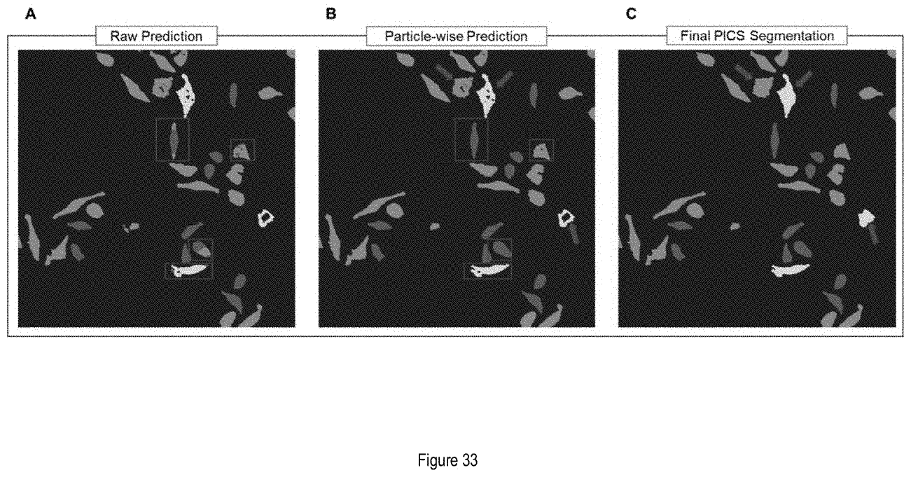

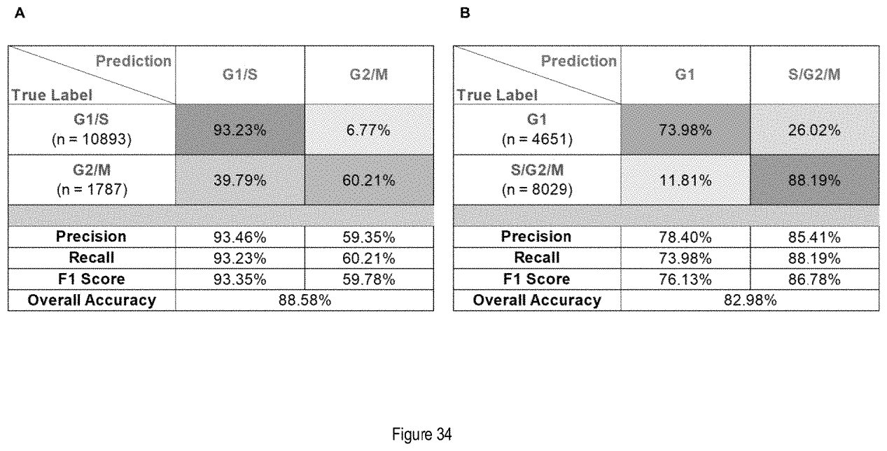

FIG. 31 shows ground truth mask generation workflow. (A) Images from the SLIM channel (left), mCherry channel (middle) and the mVenus channel (right). (B) Preliminary masks generated from the SLIM and fluorescence images using adaptive thresholding. (C) Combing three masks in (B). Holes in cell masks were removed during analysis to avoid errors in cell dry mass and area. Scale bar is 100 μm. FIG. 32 shows PICS performance evaluated at a pixel level. FIG. 33 shows post-processing workflow. (A) Raw prediction from PICS. (B) Prediction map after enforcing particle consistency and removing small particles. A few examples were shown in the red rectangles. (C) Prediction map after filling in the holes in the masks. Masks at this stage were used for analysis. FIG. 34 shows confusion matrix after merging two labels together. (A) Confusion matrix after merging “G1” and “S” into one class. (B) Confusion matrix after merging ‘S” and “G2/M” into one class.

The methods, devices, processing, and logic described above and below may be implemented in many different ways and in many different combinations of hardware and software. For example, all or parts of the implementations may be circuitry that includes an instruction processor, such as a Graphics Processing Unit (GPU), Central Processing Unit (CPU), microcontroller, or a microprocessor; an Application Specific Integrated Circuit (ASIC), Programmable Logic Device (PLD), or Field Programmable Gate Array (FPGA); or circuitry that includes discrete logic or other circuit components, including analog circuit components, digital circuit components or both; or any combination thereof. The circuitry may include discrete interconnected hardware components and/or may be combined on a single integrated circuit die, distributed among multiple integrated circuit dies, or implemented in a Multiple Chip Module (MCM) of multiple integrated circuit dies in a common package, as examples.

The circuitry may further include or access instructions for execution by the circuitry. The instructions may be embodied as a signal and/or data stream and/or may be stored in a tangible storage medium that is other than a transitory signal, such as a flash memory, a Random Access Memory (RAM), a Read Only Memory (ROM), an Erasable Programmable Read Only Memory (EPROM); or on a magnetic or optical disc, such as a Compact Disc Read Only Memory (CDROM), Hard Disk Drive (HDD), or other magnetic or optical disk; or in or on another machine-readable medium. A product, such as a computer program product, may particularly include a storage medium and instructions stored in or on the medium, and the instructions when executed by the circuitry in a device may cause the device to implement any of the processing described above or illustrated in the drawings.

The implementations may be distributed as circuitry, e.g., hardware, and/or a combination of hardware and software among multiple system components, such as among multiple processors and memories, optionally including multiple distributed processing systems. Parameters, databases, and other data structures may be separately stored and managed, may be incorporated into a single memory or database, may be logically and physically organized in many different ways, and may be implemented in many different ways, including as data structures such as linked lists, hash tables, arrays, records, objects, or implicit storage mechanisms. Programs may be parts (e.g., subroutines) of a single program, separate programs, distributed across several memories and processors, or implemented in many different ways, such as in a library, such as a shared library (e.g., a Dynamic Link Library (DLL)). The DLL, for example, may store instructions that perform any of the processing described above or illustrated in the drawings, when executed by the circuitry. Examples are listed below.

Example A: A method including:

obtaining specific quantitative image data captured via a quantitative imaging technique, the specific quantitative image data including a quantitative parameter value and a pixel value for a pixel of the specific quantitative image data, where the quantitative parameter value is derived, at least in part, from the pixel value;

determining a specific context mask for the specific quantitative image data by comparing the specific quantitative image data to previous quantitative image data for a previous sample via application of the specific quantitative image data to the input of a neutral network trained using constructed context masks generated based on the previous sample and the previous quantitative image data;

applying the specific context mask to the specific quantitative image data to determine a context value for the pixel; and

based on the pixel and the quantitative parameter value, determining a quantitative characterization for the context value.

A2. The method of example A or any of the other examples in the present disclosure, including altering the pixel value to indicate the context value.

A3. The method of example A or any of the other examples in the present disclosure, where the constructed context masks include dye-contrast images captured of the previous samples after exposure of the previous samples to a contrast dye.

A4. The method of example A3 or any of the other examples in the present disclosure, where the contrast dye includes a fluorescent material.

A4B. The method of example A3 or any of the other examples in the present disclosure, where the context value includes an expected dye concentration level at the pixel.

A5. The method of example A or any of the other examples in the present disclosure, where the constructed context masks include operator input context designations.

A6. The method of example A5 or any of the other examples in the present disclosure, where the operator input context designations indicate that portions of an image depict an instance of a particular biological structure.

A6B. The method of example A6 or any of the other examples in the present disclosure, where the context value indicates a determination that the pixel depicts, at least in part, an instance of the particular biological structure.

A7. The method of example A or any of the other examples in the present disclosure, where:

the quantitative imaging technique includes a non-destructive imaging technique; and

constructed context masks include images captured via a biologically-destructive imaging technique.

A8. The method of example A or any of the other examples in the present disclosure, where the quantitative imaging technique includes:

quantitative phase imaging;

gradient light interference microscopy;

spatial light inference microscopy;

diffraction tomography;

Fourier transform light scattering; or

any grouping of the foregoing.

A9. The method of example A or any of the other examples in the present disclosure, where obtaining the specific quantitative image data captured via a quantitative imaging technique includes capturing the pixel value via a pixel capture array positioned at a plane of a comparative effect generated by light rays traversing an objective and a processing optic.

Example B. A method including:

obtaining quantitative image data captured via quantitative imaging of a sample, the quantitative image data including multiple pixels, each of the multiple pixels including a respective quantitate parameter value;

obtaining a constructed context mask for the sample, the constructed context mask including a context value for each of the multiple pixels;

creating an input-result pair by pairing the constructed context mask as a result to an input including the quantitative image data; and

applying the input-result pair to a neural network to adjust interneuron weights within the neural network.

B2. The method of example B or any of the other examples in the present disclosure, where applying the input-result pair to a neural network includes determining a deviation from the constructed context mask by a simulated context mask at an output of the neural network when the quantitative image data is applied as an input to the neural network when a test set of interneuron weights are present within the neural network.

B3. The method of example B2 or any of the other examples in the present disclosure, where determining the deviation includes determining a loss value between the constructed context mask and the simulated context mask to quantify the deviation.

B4. The method of example B3 or any of the other examples in the present disclosure, where applying the input-result pair to a neural network to adjust interneuron weights within the neural network includes adjusting the interneuron weights to achieve a reduction in the loss function according to an optimization algorithm.

B5. The method of example B4 or any of the other examples in the present disclosure, where the optimization algorithm includes a least squares algorithm, a gradient descent algorithm, differential algorithm, a direct search algorithm, a stochastic algorithm, or any grouping thereof.

B6. The method of example B2 or any of the other examples in the present disclosure, where the neural network includes a U-net neural network to support an image transformation operation between the quantitative image data and the simulated context mask.

B7. The method of example B or any of the other examples in the present disclosure, where the constructed context mask includes a dye-contrast image captured of the samples after exposure of the samples to a contrast dye.

B8. The method of example B7 or any of the other examples in the present disclosure, where the contrast dye includes a fluorescent material.

B9. The method of example B or any of the other examples in the present disclosure, where the constructed context mask includes operator input context designations.

B10. The method of example B9 or any of the other examples in the present disclosure, where the operator input context designations indicate that portions of the quantitative image data depict an instance of a particular biological structure.

B11. The method of example B or any of the other examples in the present disclosure, where:

the quantitative imaging includes a non-destructive imaging technique; and

constructed context mask includes an image captured via a biologically-destructive imaging technique.

B12. The method of example B or any of the other examples in the present disclosure, where the quantitative imaging includes:

quantitative phase imaging;

gradient light interference microscopy;

spatial light inference microscopy;

diffraction tomography;

Fourier transform light scattering; or

any grouping of the foregoing.

Example C. A biological imaging device including:

a capture subsystem including:

an objective;

a processing optic positioned relative to the objective to generate a comparative effect from a light ray captured through the objective;

a pixel capture array positioned at a plane of the comparative effect;

a processing subsystem including:

memory configured to store:

raw pixel data from the pixel capture array; and

computed quantitative parameter values for pixels of the raw pixel data;

a neural network trained using constructed structure masks generated based on previous quantitative parameter values and previous pixel data;

a computed structure mask for the pixels;

a structure integrity index;

a processor in data communication with memory, the processor configured to:

determine the computed quantitative parameter values for the pixels based on the raw pixel data and the comparative effect;

via execution of the neural network, determine the computed structure mask by assigning a subset of the pixels that represent portions of a selected biological structure identical mask values within the computed structure mask;

based on ones of the computed quantitative parameter values corresponding to the subset of the pixels, determine a quantitative characterization of the selected biological structure; and

reference the quantitative characterization against the structure integrity index to determine a condition of the selected biological structure.

C2. The biological imaging device of example C or any of the other examples in the present disclosure, where:

the biological imaging device includes an assistive-reproductive-technology (ART) imaging device; and

the biological structure includes a structure within a gamete, a zygote, a blastocyst, or any grouping thereof; and

optionally, the condition includes a predicted success rate for zygote cleavage or other reproductive stage.

Example D. A device including:

memory configured to store:

specific quantitative image data for pixels of the pixel data captured via a quantitative imaging technique, the specific quantitative image data including a quantitative parameter value and a pixel value for a pixel of the specific quantitative image data, where the quantitative parameter value is derived, at least in part, from the pixel value;

a neutral network trained using constructed context masks generated based on a previous sample and a previous quantitative image data, the previous quantitative image data captured by preforming the quantitative imaging technique on the previous sample; and

a computed structure mask for the pixels;

a processor in data communication with memory, the processor configured to:

obtain the specific quantitative image data captured via a quantitative imaging technique, the specific quantitative image data including a quantitative parameter value and a pixel value for a pixel of the specific quantitative image data, where the quantitative parameter value is derived, at least in part, from the pixel value;

determine a specific context mask for the specific quantitative image data by comparing the specific quantitative image data to previous quantitative image data by applying the specific quantitative image data to the input of the neutral network;

apply the specific context mask to the specific quantitative image data to determine a context value for the pixel; and

based on the pixel and the quantitative parameter value, determine a quantitative characterization for the context value.

Example E. A device to implement the method of any example in the present disclosure.

Example F. A method implemented by operating the device of any of the examples in the present disclosure.

Example G. A system configured to implement any of or any combination of the features described in the specification and/or the examples in the present disclosure.

Example H. A method including implementing any of or any combination of the features described in the specification and/or the examples in the present disclosure.

Example I. A product including:

machine-readable media;

instructions stored on the machine-readable media, the instructions configured to cause a machine to implement any of or any combination of the features described in the specification and/or the examples in the present disclosure.

Example J. The product of example I, where:

the machine-readable media is other than a transitory signal; and/or

the instructions are executable.

Example K1. A method including:

obtaining specific quantitative imaging data (QID) corresponding to an image of a biostructure;

determining a context spectrum selection from context spectrum including a range of selectable values by:

comparing the specific QID to previous QID by applying the specific QID to an input layer of a context-spectrum neural network, the context-spectrum neural network including:

a naive layer trained using an imparted learning process based on the the previous QID and constructed context spectrum data generated based on a previous image associated with the previous QID;

an instructed layer including imported intermural weights obtained through a transfer learning process from a precursor neural network trained using multiple different image transformation tasks;

mapping the context spectrum selection to the image to generate a context spectrum mask for the image; and

based on the context spectrum mask determining a condition of the biostructure, where:

optionally, the method is according to the method of any of the other examples in the present disclosure.

Example K2. A method including:

obtaining specific quantitative imaging data (QID) corresponding to an image;

determining a context spectrum selection from context spectrum including a range of selectable values by:

comparing the specific QID to previous QID by applying the specific QID to an input layer of a neural network, the neural network including:

a naive layer trained using an imparted learning process based on the the previous QID and constructed context spectrum data generated based on a previous image associated with the previous QID;

an instructed layer including imported intermural weights obtained through a transfer learning process from a precursor neural network trained using multiple different image transformation tasks;

mapping the context spectrum selection to the image to generate a context spectrum mask for the image, where:

optionally, the method is according to the method of any of the other examples in the present disclosure.

Example K3. The method of any example in the present disclosure, where the precursor neural network includes a neural network trained using input images and output image pairs constructed using multiple classes of image transformations, optionally including:

an image filter effect;

an upsampling/downsampling operation;

a mask application for one-or-more-color masks;

an object removal;

a facial recognition;

an image overlay;

a lensing effect;

a mathematical transform;

a re-coloration operation;

a selection operation;

a biostructure identification;

a biometric identification; and/or

other image transformation tasks.

Example K4. The method of any example in the present disclosure, where the transfer learning process includes copying the instructed layer from the precursor neural network, where optionally:

the instructed layer includes a hidden layer (a layer between the input and output layers) from the precursor neural network.

Example K5. The method of any example in the present disclosure, where the context-spectrum neural network includes an EfficientNet Unet, where optionally, the EfficientNet Unet includes one or more first layers for adapting a vector size to operational size for another layer of the EfficientNet Unet.

Example K6. The method of any example in the present disclosure, where the biological structure includes cells, tissue, cell parts, organs, HeLa cells, and/or other biological structures.

Example K7. The method of any example in the present disclosure, where the condition includes viability, cell membrane integrity, health, or other biological status.

Example K8. The method of any example in the present disclosure, where context spectrum includes a continuum or near continuum of selectable states.

Example K9. The method of any example in the present disclosure, where the context spectrum selectable multiple levels of predicted dye diffusion.

Example K10. The method of any example in the present disclosure, where the imparted learning process includes training the layers of the context-spectrum neural network using the previous QID and corresponding constructed images, e.g., without transfer learning for the naive layer.

Example K11. The method of any example in the present disclosure, where the context-spectrum neural network is assembled to include the naive and instructed layers and trained using the imparted learning process after assembly.

Example K12. The method of any example in the present disclosure, where the constructed context spectrum data includes ground truth health states for cells, where:

optionally, the ground truth health states including a viable state, an injured state, and a dead state; and

optionally, the context spectrum selection directly indicates a condition of the biological structure without additional analysis.

### Example Implementations

The example implementations below are intended to be illustrative examples of the techniques and architectures discussed above. The example implementations are not intended to constrain the above techniques and architectures to particular features and/or examples but rather demonstrate real world implementations of the above techniques and architectures. Further, the features discussed in conjunction with the various example implementations below may be individually (or in virtually any grouping) incorporated into various implementations of the techniques and architectures discussed above with or without others of the features present in the various example implementations below.

Artificial intelligence (AI) can transform one form of contrast into another. Various example implementations include phase imaging with computational specificity (PICS), which includes a combination of quantitative phase imaging and AI, which provides quantitative information about unlabeled live cells with high specificity. In various example implementations, an imaging system allows for automatic training, while inference is built into the acquisition software and runs in real-time. In certain embodiments of the present disclosure, by applying computed specificity maps back to QPI data, the growth of both nuclei and cytoplasm may be measured independently, over many days, without loss of viability. In various example implementations, using a QPI method that suppresses multiple scattering, the dry mass content of individual cell nuclei within spheroids may be measured.

The ability to evaluate sperm at the microscopic level, using high throughput would be useful for assisted reproductive technologies (ART), as it can allow specific selection of sperm cells for in vitro fertilization (IVF). The use of fluorescence labels has enabled new cell sorting strategies and given new insights into developmental biology.

In various example implementations, a trained a deep convolutional neural network to performs semantic segmentation on quantitative phase maps. This approach, a form of phase imaging with computational specificity, allows analyzation thousands of sperm cells and identify correlations between dry mass content and artificial reproduction outcomes. Determination of the dry mass content ratios between the head, midpiece, and tail of the sperm cells can be used to predict the percentages of success for zygote cleavage and embryo blastocyst rate.

The high incidence of human male factor infertility suggests a need for examining new ways of evaluating male gametes. Certain embodiments of the present disclosure provide a new approach that combines label-free imaging and artificial intelligence to obtain nondestructive markers for reproductive outcomes. The phase imaging system reveals nanoscale morphological details from unlabeled cells. Deep learning provides a specificity map segmenting with high accuracy the head, midpiece, and tail. Using these binary masks applied to the quantitative phase images, the dry mass content of each component was measure precisely. The dry mass ratios represent intrinsic markers with predictive power for zygote cleavage, and embryo blastocyst development.

Various example implementations include phase imaging with computational specificity in which QPI and A1 are combined to infer quantitative information from unlabeled live cells, with high specificity and without loss of cell viability.

Various example implementations include a microscopy concept, referred to as phase imaging with computational specificity (PICS), in which the process of learning is automatic and retrieving computational specificity is part of the acquisition software, performed in real-time. In various example implementations, deep learning is applied to QPI data, generated by SLIM (spatial light interference microscopy) and GLIM (gradient light interference microscopy). In some cases, these systems may use white-light and common-path setups and, thus, provide high spatial and temporal sensitivity. Because they may be add-ons to existing microscopes and are compatible with the fluorescence channels, these systems provide simultaneous phase and fluorescence images from the same field of view. As a result, the training data necessary for deep learning is generated automatically, without the need for manual annotation. In various example implementations, QPI may replace some commonly used tags and stains and eliminate inconveniences associated with chemical tagging. This is demonstrated in real world examples with various fluorescence tags and operations on diverse cell types, at different magnifications, on different QPI systems. Combining QPI and computational specificity allows us to quantify the growth of subcellular components (e.g. nucleus vs cytoplasm) over many cell cycles, nondestructively. Using GLIM, spheroids where imaged, which demonstrates that PICS can perform single-cell nucleus identification even in such turbid structures.

In various example implementations, PICS performs automatic training by recording both QPI and fluorescence microscopy of the same field of view, on the same camera, with minimal image registration. The two imaging channels are integrated seamlessly by the software that controls both the QPI modules, fluorescence light path, and scanning stage. The PICS instrument can scan a large field of view, e.g., entire microscope slides, or multi-well plates, as needed. PICS can achieve multiplexing by automatically training on multiple fluorophores and performing inference on single-phase image. PICS performs real-time inference, because the A1 code may be implemented into the live acquisition software. The computational inference is faster than the image acquisition rate in SLIM and GLIM, which is up to 15 frames per second, thus, specificity is added without noticeable delay. To the microscope user, it may be difficult to state whether the live image originates in a fluorophore or the computer GPU. Using the specificity maps obtained by computation, the QPI channel is exploited to compute the dry mass density image associated with the particular subcellular structures. For example, using this procedure, a previously unachievable task was demonstrated: the measurement of growth curves of cell nuclei vs. cytoplasm over several days, nondestructively. Using a QPI method dedicated to imaging 3D cellular systems (GLIM), subcellular specificity may be added into turbid structures such as spheroids.

In a proof-of-concept example, use an inverted microscope (Axio Observer Z1, Zeiss) equipped with a QPI module (CellVista SLIM Pro and CellVista GLIM Pro, Phi Optics, Inc.). Other microscope systems may be used. The microscope is programmed to acquire both QPI and fluorescence images of fixed, tagged cells. Once the microscope “learned” the new fluorophore, PICS can perform inference on the live, never labeled cells. Due to the absence of chemical toxicity and photobleaching, as well as the low power of the white light illumination, PICS can perform dynamic imaging over arbitrary time scales, from milliseconds to weeks, without cell viability concerns. Simultaneous experiments involving multi-well plates can be performed to assay the growth and proliferation of cells of specific cellular compartments. The inference is implemented within the QPI acquisition time, such that PICS performs in real-time.

PICS combines quantitative measurements of the object's scattering potential with fluorescence microscopy. The GLIM module controls the phase between the two interfering fields outputted by a DIC microscope. four intensity images corresponding to phase shifts incremented in steps of π/2 were acquired and these were combined to obtain a quantitative phase gradient map. This gradient is integrated using a Hilbert transform method, as described in. The same camera records fluorescence images via epi-illumination providing a straightforward way to combine the fluorescence and phase images.

In various example implementations, co-localized image pairs (e.g., input-result pairs) are used to train a deep convolutional neural network to map the label-free phase images to the fluorescence data. For deep learning, a variant of U-Net with three modifications may be used. A batch normalization layers before all the activation layers is added, which helps accelerate the training. The number of parameters in the network may be reduced by changing the number of feature maps in each layer of the network to a quarter of the original size. This change reduced GPU memory usage during training, without loss of performance. The modified U-Net model used approximately 1.9 million parameters, while another implementation had over 30 million parameters.

Residual learning was implemented with the hypothesis that it is easier for the models to approximate the mapping from phase images to the difference between phase images and fluorescence images. Thus, an add operation between the input and the output of the last convolutional block to generate the final prediction was added.

In various example implementations, high fidelity digital stains can be generated from as few as 20 image pairs (roughly 500 sample cells).

Because of the nondestructive nature of PICS, it may be applied to monitor cells over extended periods, of many days, without a noticeable loss in cell viability. In order to demonstrate a high content cell growth screening assay, unlabeled SW480 and SW620 cells were imaged over seven days and PICS predicted both DAPI (nucleus) and DIL (cell membrane) fluorophores. The density of the cell culture increased significantly over the seven-day period, a sign that cells continued their multiplication throughout the duration of imaging. PICS can multiplex numerous stain predictions simultaneously, as training can be performed on an arbitrary number of fluorophores for the same cell type. Multiple networks can be evaluated in parallel on separate GPUs.

PICS-DIL may be used to generate a binary mask, which, when applied to the QPI images, yields the dry mass of the entire cell. Similarly, PICS-DAPI allows the nuclear dry mass to be obtained. Thus, the dry mass content of the cytoplasm and nucleus can be independently and dynamically monitored.

GLIM may extend QPI applications to thicker, strongly scattering structures, such as embryos, spheroids, and acute brain slices. GLIM may improves image quality by suppressing artifacts due to multiple scattering and provides a quantitative method to assay cellular dry-mass. PICS can infer the nuclear map with high accuracy. A binary mask using PICS and DAPI images was created. The fraction of mass found inside the two masks was compared. In the example proof-of-concept, the average error between inferring nuclear dry mass based on the DAPI vs. PICS mask is 4%.

In various example implementations, by decoupling the amplitude and phase information, QPI images outperform their underlying modalities (phase contrast, DIC) in A1 tasks. This capability is showcased in GLIM which provides high-contrast imaging of thick tissues, enabling subcellular specificity in strongly scattering spheroids.

In various example implementations, SLIM uses a phase-contrast microscope in a similar way to how GLIM used DIC. SLIM uses a spatial light modulator matched to the back focal plane of the objective to control the phase shift between the incident and scattered components of the optical field. Four such phase-contrast like frames may be recorded to recover the phase between the two fields. The total phase is obtained by estimating the phase shift of the transmitted component and compensating for the objective attenuation. The “halo” associated with phase-contrast imaging is corrected by a non-linear Hilbert transform-based approach.

In various example implementations, while SLIM may have higher sensitivity, the GLIM illumination path may perform better in some strongly scattering samples and dense well plates. In strongly scattering samples, the incident light, which acts as the reference field in SLIM, vanishes exponentially. In dense microplates, the transmitted light path is distorted by the meniscus or blocked by high wall.

In various example implementations, a hardware backend may implement TensorRT (NVIDIA) to support real-time inference. In an example GLIM system, the phase shift is introduced by a liquid crystal variable retarder, which takes approximately 70 ms to fully stabilize. In an example implementation, SLIM system a ring pattern is written on the modulator and 20 ms is allowed for the crystal to stabilize. Next, four such intensity images are collated to reconstruct the phase map. In GLIM, the image is integrated and in SLIM the phase-contrast halo artifact (is removed. The phase map is then passed into a deep convolution neural network based on the U-Net architecture to produce a synthetic stain. The two images are rendered as an overlay with the digital stain superimposed on the phase image. In the “live” operating mode used for finding the sample and testing the network performance, a PICS image is produced for every intensity frame. In various example implementations, the rate-limiting factor is the speed of image acquisition rather than computation time.

The PICS system may use a version of the U-Net deep convolutional neural architecture to translate the quantitative phase map into a fluorescence one. To achieve real-time inference, TensorRT (NVIDIA) may be which automatically tunes the network for the specific network and graphics processing unit (GPU) pairings.

In various example implementations, the PICS inference framework is designed to account for differences between magnification and camera frame size. Differences in magnification are accounted for by scaling the input image to the networks' required pixel size using various libraries, such as NVIDIA's Performance Primitives library. To avoid tuning the network for each camera sensor size, an optimized network for the largest image size and extend smaller images by mirror padding may be created. To avoid the edge artifacts typical of deep convolutional neural networks, a 32-pixel mirror pad may be performed for inferences.

In various example implementations, a neural network with a U-Net architecture, which effectively captures the broad features typical of quantitative phase images, may be used. Networks were built using TensorFlow and Keras, with training performed on a variety of computers including workstations (NVIDIA GTX 1080 & GTX 2080) as well as isolated compute nodes (HAL, NCSA, 4×NVIDIA V100). Networks were trained with the adaptive moment estimator (ADAM) against a mean squared error optimization criterion.

Phase and fluorescence microscopy images, I(x,y), were normalized for machine learning as

$${I}_{ml input} ( x , y ) = med ( 0 , \frac{I ( x , y ) - {ρ}_{min}}{{ρ}_{max} - {ρ}_{min}} , 1 )$$

where ρₘᵢₙ and ρₘₐₓ are the minimum, and maximum pixel values across the entire training set, and med is a pixel-wise median filter designed to bring the values within the range [0,1]. Spatio-temporal broadband quantitative phase images exhibit strong sectioning and defocus effects. To address focus related issues, images were acquired as a tomographic stack. In various example implementations, the Haar wavelet criterion from may be used to select the three most in-focus images for each mosaic tile.

The SW480 and SW620 pairing is a popular model for cancer progression as the cells were harvested from the tumor of the same patient before and after a metastasis event. Cells were grown in Leibovitz's L-15 media with 10% FBS and 1% pen-strep at atmospheric CO₂. Mixed SW cells were plated at a 1:1 ratio at approximately 30% confluence. The cells were then imaged to demonstrate that the various example implementations may be used for imaging in real-world biological applications as discussed in U.S. Provisional Application No. 62/978,194, which was previously incorporated by reference.

In various example implementations, highly sensitive QPI in combination with deep learning allows us to identification subcellular compartments of unlabeled bovine spermatozoa. The deep learning semantic segmentation model automatically segments the head, midpiece, and tail of individual cells. These predictions may be used to measure the respective dry mass of the components. The relative mass content of these components correlates with the zygote cleavage and embryo quality. The dry mass ratios, i.e. head/midpiece (H/M), head/tail (H/T), midpiece/tail (M/T), can be used as intrinsic markers for reproductive outcomes.

To image the unlabeled spermatozoa SLIM, or other QI techniques, may be used. Due to the white light illumination, SLIM lacks speckles, which yields sub-nanometer pathlength spatial sensitivity.

A representative sperm cell may be reconstructed from a series of through-focus measurements (z-stack). Various cellular compartments may be revealed with high resolution and contrast. The highest density region of the sperm is the mitochondria-rich neck (or midpiece), which is connected to a denser centriole vault leading to the head. Inside the head, the acrosome appears as a higher density sheath surrounding a comparably less optically dense nucleus. The posterior of the sperm consists of a flagellum followed by a less dense tail.

The training data were annotated manually by individuals trained to identify the sperm head, midpiece, and tail. A fraction of the tiles was manually segmented by one annotator using ImageJ. The final segmentations were verified by a second annotator. In In an example implementation, for the sperm cells, the sharp discontinuity between the background and cell was traced, separated by an abrupt change in refractive index. As a proof-of-concept and to reduce computing requirements, images were down-sampled to match the optical resolution. To account for the shift variance of all convolutional neural networks, the data were augmented by a factor of 8, using rotation, flipping, and translation. To improve the segmentation accuracy, a two-pass training procedure where an initial training round was corrected and used for a second, final round was used. Manual annotation for the second round is comparably fast, and mostly for debris and other forms of obviously defective segmentation were corrected. The resulting semantic segmentation maps were applied to the phase image to compute the dry mass content of each component. By using a single neural network, rather than a group of annotators, differences in annotation style can be compensated. In the example implementation, training and inference were performed on twenty slides.

For semantic segmentation, in the example implementation, a U-Net based deep convolution neural network was used. The last sigmoid layer in the U-Net with a softmax layer, which predicts the class probability of every single pixel in the output layer, is replaced. The final segmentation map can be obtained by applying an argmax function on the neural network output. The model is trained using categorical cross entropy loss and Adam Optimizer. The model was trained with a learning rate of 5e-6 and a batch size of 1 for 30 epochs. Within each epoch, the model was given 3,296 image pairs for weight update. The model attained an F1-score of over 0.8 in all four classes. Once the model is trained, the weights are ported into the imaging software.

The dry mass ratios between the head, midpiece, and tail were measured, rather than the absolute dry mass, for which there were no statistically significant correlations.

The results from the proof-of-concept suggest that a long tail is beneficial. However, when the embryo blastocyst development rate is evaluated, it appears that a large H/M value is desirable, while the other two ratios are only weakly correlated. This result appears to indicate that a denser head promotes embryo blastocyst development. Note that this subgroup of spermatozoa that are associated with the embryo blastocyst development rate have, with a high probability, large tails.

Having a head or midpiece with relatively more dry mass penalizes early stages of fertilization (zygote cleavage, negative trend) while having a larger head relative to midpiece is important for embryo development (blastocyst rate, positive trend).

Various example implementations would be useful when selecting among seemingly healthy sperm, with no obvious defects. Various example implementations may be used for automating the annotation of a large number of cells.

IVF clinics have been using phase contrast microscopes for nondestructive observation. In various example implementations, PICS can be implemented to these existing systems as an add-on.

Deep Learning

In various example implementations, the task may be formulated as a 4-class semantic segmentation problem and adapted from the U-Net architecture. The example model may take as input a SLIM image of dimension 896×896 and produce a 4-channel probability distribution map, one for each class (head, neck, tail and background). An argmax function is then applied on this 4-channel map to obtain the predicted segmentation mask. The model is trained with categorical cross entropy loss and the gradient is computed using Adam optimizer. The model may be trained with a learning rate of 5e-6 for 30 epochs. The batch size is set to 1, but may be increased with greater GPU memory availability. Within each epoch, the model weights were updated 3296 steps as each image is augmented 8 times.

$$E = \frac{1}{r \times c} \cdot {\sum }_{r = 1}^{h} {\sum }_{c = 1}^{w} {\sum }_{k = 1}^{4} [ δ ( y [ r ] [ c ] == k ) \cdot log ( \hat{y} [ r ] [ c ] [ k ] ) ]$$

The trained model was run on the test set and recorded the confusion matrix. To understand the performance of the model, precision, recall and F-1 score were utilized.

$$\begin{matrix}Precision = \frac{True Positive}{Predicted Positive} = \frac{True Positive}{True Positive + False Positive} \\ Recall = \frac{True Positive}{Labeled Positive} = \frac{True Positive}{True Positive + False Negative} \\ F 1 = \frac{2}{\frac{1}{Precision} + \frac{1}{Recall}} = \frac{2 \cdot Precision \cdot Recall}{Precision + Recall}\end{matrix}$$

The model achieved over 0.8 F-1 score on all four classes.

Once the model is trained, the kernel weights were transposed using a python script into the TensorRT-compatible format. The exact same network architecture was constructed using TensorRT C++ API and loaded the trained weights. This model was then constructed on GPU and optimized layer-by-layer via TensorRT for best inference performance.

The model based on the modified U-Net architecture discussed above was trained for 100 epochs with a learning rate of 1e-4. The model also achieved over 0.8 F1-Score for all four classes. In particular, it reached 0.94 F1-Score for segmenting the head.

Various implementations have been specifically described. However, many other implementations are also possible.

While the particular disclosure has been described with reference to illustrative embodiments, this description is not meant to be limiting. Various modifications of the illustrative embodiments and additional embodiments of the disclosure will be apparent to one of ordinary skill in the art from this description. Those skilled in the art will readily recognize that these and various other modifications can be made to the exemplary embodiments, illustrated and described herein, without departing from the spirit and scope of the present disclosure. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

What is claimed is:

1. A method comprising:
obtaining specific quantitative imaging data (QID) corresponding to an image of a biostructure;
determining a context spectrum selection from context spectrum including a range of selectable values by:
applying the specific QID to an input layer of a context-spectrum neural network, wherein the context-spectrum neural network is trained, according to a combination of focal loss and dice loss, based on previous QID and constructed context spectrum data associated with the previous QID;
mapping the context spectrum selection to the image to generate a context spectrum mask for the image; and
determining a condition of the biostructure based on the context spectrum mask.

2. The method according to claim 1, wherein:
the previous QID are obtained corresponding to an image of a second biostructure; and
the constructed context spectrum data comprises a ground truth condition of the second biostructure.

3. The method according to claim 1, wherein:
the context-spectrum neural network comprises an EfficientNet Unet comprising one or more first layers for adapting a vector size to operational size for another layer of the EfficientNet Unet.

4. The method according to claim 1, wherein:
the biostructure comprises at least one of the following: a cell, a tissue, a cell part, an organ, or a HeLa cell.

5. The method according to claim 1, wherein:
the condition of the biostructure comprises at least one of the following: viability, cell membrane integrity, health, or cell cycle.

6. The method according to claim 1, wherein:
the context spectrum comprises a continuum or near continuum of selectable states.

7. The method according to claim 1, wherein:
the condition of the biostructure comprises one of a viable state, an injured state, or a dead state; or
the condition of the biostructure comprises one of a cell growth stage (G1 phase), a deoxyribonucleic acid (DNA) synthesis stage (S phase), or a cell growth/mitotic stage (G2/M phase).

8. An apparatus, comprising:
a memory storing instructions; and
a processor in communication with the memory, wherein, when the processor executes the instructions, the processor is configured to cause the apparatus to perform:
obtaining specific quantitative imaging data (QID) corresponding to an image of a biostructure;
determining a context spectrum selection from context spectrum including a range of selectable values by:
applying the specific QID to an input layer of a context-spectrum neural network, wherein the context-spectrum neural network is trained, according to a combination of focal loss and dice loss, based on previous QID and constructed context spectrum data associated with the previous QID;
mapping the context spectrum selection to the image to generate a context spectrum mask for the image; and
determining a condition of the biostructure based on the context spectrum mask.

9. The apparatus according to claim 8, wherein:
the previous QID are obtained corresponding to an image of a second biostructure; and
the constructed context spectrum data comprises a ground truth condition of the second biostructure.

10. The apparatus according to claim 8, wherein:
the context-spectrum neural network comprises an EfficientNet Unet comprising one or more first layers for adapting a vector size to operational size for another layer of the EfficientNet Unet.

11. The apparatus according to claim 8, wherein:
the biostructure comprises at least one of the following: a cell, a tissue, a cell part, an organ, or a HeLa cell.

12. The apparatus according to claim 8, wherein:
the condition of the biostructure comprises at least one of the following: viability, cell membrane integrity, health, or cell cycle.

13. The apparatus according to claim 8, wherein:
the context spectrum comprises a continuum or near continuum of selectable states.

14. The apparatus according to claim 8, wherein:
the condition of the biostructure comprises one of a viable state, an injured state, or a dead state; or
the condition of the biostructure comprises one of a cell growth stage (G1 phase), a deoxyribonucleic acid (DNA) synthesis stage (S phase), or a cell growth/mitotic stage (G2/M phase).

15. A non-transitory computer readable storage medium storing computer readable instructions, wherein, the computer readable instructions, when executed by a processor, are configured to cause the processor to perform:
obtaining specific quantitative imaging data (QID) corresponding to an image of a biostructure;
determining a context spectrum selection from context spectrum including a range of selectable values by:
applying the specific QID to an input layer of a context-spectrum neural network, wherein the context-spectrum neural network is trained, according to a combination of focal loss and dice loss, based on previous QID and constructed context spectrum data associated with the previous QID;
mapping the context spectrum selection to the image to generate a context spectrum mask for the image; and
determining a condition of the biostructure based on the context spectrum mask.

16. The non-transitory computer readable storage medium according to claim 15, wherein:
the previous QID are obtained corresponding to an image of a second biostructure; and
the constructed context spectrum data comprises a ground truth condition of the second biostructure.

17. The non-transitory computer readable storage medium according to claim 15, wherein:
the context-spectrum neural network comprises an EfficientNet Unet comprising one or more first layers for adapting a vector size to operational size for another layer of the EfficientNet Unet.

18. The non-transitory computer readable storage medium according to claim 15, wherein:
the biostructure comprises at least one of the following: a cell, a tissue, a cell part, an organ, or a HeLa cell.

19. The non-transitory computer readable storage medium according to claim 15, wherein:
the condition of the biostructure comprises at least one of the following: viability, cell membrane integrity, health, or cell cycle.

20. The non-transitory computer readable storage medium according to claim 15, wherein:
the condition of the biostructure comprises one of a viable state, an injured state, or a dead state; or
the condition of the biostructure comprises one of a cell growth stage (G1 phase), a deoxyribonucleic acid (DNA) synthesis stage (S phase), or a cell growth/mitotic stage (G2/M phase).
